(19) United States
(12) Patent Application Publication
(10) Pub. No.: US 2002/0081731 A1
(43) Pub. Date: Jun. 27, 2002

(54) MANIPULATION OF PLANT CELL AND TISSUE CULTURES

(76) Inventors: ANGELA STAFFORD, CASTLETON (GB); MALCOLM MORVILLE, SHREWSBURY, MA (US)

Correspondence Address: PAUL T CLARK, CLARK AND ELBING, 176 FEDERAL STREET, BOSTON, MA, 02110

(21) Appl. No.: 08/887,532

(22) Filed: Jul. 3, 1997

Related U.S. Application Data
(63) Continuation of application No. 08/444,846, filed on May 19, 1995, now abandoned.

(51) Int. Cl. C12N 5/00; C12N 5/02

(52) U.S. Cl. 435/420; 435/410; 435/430

(57) ABSTRACT

Methods of affecting secondary metabolite production and secondary metabolite production profiles in plant cell and tissue cultures with DNA methylation inhibitors and elicitor systems.

### BACKGROUND OF THE INVNETION

The invention relates to methods of manipulating plant cell and plant tissue cultures to affect phytochemical production and growth characteristics. General methods of plant cell or tissue culturing include steps such as germinating a seed, initiating a callus from explant tissue, maintaining a callus by subculture, initiating a liquid culture such as a suspension culture, and maintaining a liquid culture by subculture. Such general procedures of plant cell and tissue culture methods are well known. Representative texts include Plant Cell Culture, A Practical Approach (Ed. R. A. Dixon) IRL Press, Oxford, Wash., (1985) and Plant Cell and Tissue Culture (Eds., A. Stafford and G. Warren) Open University Press, Milton, Keynes (1991).

Plant cells produce endogenous elicitors such as pectic fragments and oligogalacturonic acids in response to environmental stresses, such as disease or damage. Some inducible phytochemicals or secondary metabolites are linked to a plant or plant cell defense mechanism. A plant cell culture can be artificially induced to produce one or more phytochemicals by exposure to an elicitor. In addition, environmental changes such as ultra-violet light and culture dilution can also stimulate production of secondary metabolites. Culture dilution includes subculturing by volume, i.e., inoculating a precise volume of culture into an excess of fresh plant culture medium.

Secondary metabolites include a diverse array of chemically unrelated compounds such as acetylenes, thiophenes, glycosides, glucosinates, purines, pyrimidines, alkaloids, phenolics (e.g., quinones), essential oils, glycosides, terpenoids (e.g., iridoids, sesquiterpenes, diterpenoids, and triterpenoids), lignans, and flavonoids. In addition, secondary metabolites include small molecules (i.e., having a molecuar weight less than 600, e.g., less than 500, or less than 400), such as substituted heterocycles. These heterocycles may be monocyclic or polycyclic, fused or bridged.

### SUMMARY OF THE INVNETION

The invention relates to methods of affecting secondary metabolite production in a plant cell or tissue culture. Affecting secondary metabolite production as used herein includes (1) increasing or decreasing production levels of phytochemicals detectable in controls; (2) causing production of detectable levels of novel or previously undetected phytochemicals; and (3) a combination of (1) and (2). Affecting a secondary metabolite production profile as used herein includes creating the potential for (1)-(3) by manipulation, (e.g., using demethylation agents) but such a method does not necessarily include a step of triggering actual phytochemical production (i.e., elicitation). Secondary metabolite production includes both intracellular production and extracellular production (e.g., phytochemical production in the medium).

In one aspect, the invention relates to methods of affecting secondary metabolite production in a plant culture. These methods include the steps of: (a) exposing a liquid plant culture to a first DNA methylation inhibitor; (b) subculturing said DNA methylation inhibitor-exposed liquid plant culture; (c) exposing said subculture to an elicitor system; and (d) maintaining said elicitor system-exposed liquid culture. The novelty of this aspect of the invention resides, in part, in not only an elicitor system (especially elicitor systems having multiple elicitors, i.e., 2, 3, or more) but also the combined steps of exposing a culture to a DNA methylation inhibitor, further subculturing, and exposing the subculture to an elicitor system.

One embodiment of the above methods includes, after step (b) and before step (c), the further step of exposing the subculture to a second DNA methylation inhibitor. Additional embodiments include methods wherein the liquid plant culture is a plant cell suspension culture or the liquid culture is a differentiated plant cell liquid culture (e.g., embryo, root, shoot, hairy root, and teratoma). Further embodiments include methods wherein: each of the first and second DNA methylation inhibitors is independently selected from 5-azacytidine, 5-aza-2′-deoxycytidine, 5-fluorocytidine, pseudoisocytidine, DL-ethionine, and 2-amino-5-ethoxy carbonylpyrimidine-4(3H) one; each of the first and second DNA methylation inhibitors is 5-aza-cytidine; the elicitor system has at least one elicitor, each elicitor being independently selected from microorganism-derived elicitors, plant-derived elicitors, and chemically-defined elicitors (e.g., independently selected from methyl jasmonate, salicylic acid, glutathione, 2,6-dichloroisonicotinic acid, cellulase, chitosan, chitin, nigeran, arachidonic acid, peroxide cascade intermediates, and elicitors derived from Candida albicans, Saccharomyces cerevisiae, Aspergillus niger, Phytophthora cryptogea, Pseudomonas syringae, and Erwinia caratovora pv. carotovora); the subculturing step (b) involves subculturing the liquid culture at least twice; and combinations of the above.

In another aspect, the invention provides a method of affecting secondary metabolite production profiles in a plant culture, including the steps of: (a) exposing an ungerminated seed to a first DNA methylation inhibitor; (b) deriving tissue from the DNA methylation inhibitor-exposed seed; (c) initiating a callus culture from the derived tissue; (d) subculturing the initiated callus culture; (e) initiating suspension from the callus subculture; and (f) maintaining the initiated suspension culture. The novelty of this aspect of the invention resides, in part, in the combination of (i) exposing an ungerminated seed with a DNA methylation inhibitor and (ii) initiating a suspension culture with cells derived from the pretreated seed.

In one embodiment of this aspect, the method further involves, after the initiating step (e), the step of exposing a subculture of the initiated suspension culture to a second DNA methylation inhibitor. The first and second DNA methylation inhibitors are independently selected from 5-azacytidine, 5-aza-2′-deoxycytidine, 5-fluorocytidine, pseudoisocytidine, DL-ethionine, and 2-amino-5-ethoxycarbonylpyrimidine-4(3H) one. Additionally, embodiments of this aspect include methods wherein: each of the first and second DNA methylation inhibitors is 5-azacytidine; the exposing step (a) involves soaking the ungerminated seed in a solution of 5-azacytidine having a concentration between 3×10⁻⁶ and 3×10⁻⁴ M; the subculturing step (d) includes subculturing the callus subculture at least two times; the maintaining step (f) includes subculturing the suspension culture at least five times; the maintaining step (f) involves subculturing the suspension culture at least ten times; the method further includes after step (f) the step of exposing the suspension culture to an elicitor system; the elicitor system has at least one elicitor, each elicitor being independently selected from microorganism-derived elicitors, plant-derived elicitors, and chemically-defined elicitors; and combinations of the above.

Another aspect of the invention features a method of affecting secondary metabolite production in a plant culture, including the steps of: (a) exposing an ungerminated seed to a first DNA methylation inhibitor; (b) deriving tissue from the DNA methylation inhibitor-exposed seed; (c) initiating a culture from the derived tissue; (d) exposing a subculture derived from the initiated culture to an elicitor system; and (e) maintaining the elicitor-exposed subculture.

One embodiment of this aspect includes after the initiating step (c) the further step of exposing a subculture derived from the initiated culture to a second DNA methylation inhibitor. As in all aspects and embodiments of the invention, each of the first and second (if any) DNA methylation inhibitors is independently selected from 5-azacytidine, 5-aza-2′-deoxycytidine, 5-fluorocytidine, pseudoisocytidine, DL-ethionine, and 2-amino-5-ethoxycarbonylpyrimidine-4(3H) one, and any other DNA methylation inhibitors, sometimes known as DNA demethylators, known to those in the art. Additionally, other embodiments include methods wherein: each of the first and second DNA methylation inhibitors is 5-azacytidine; the exposing step includes soaking the ungerminated seed in a solution of 5-azacytidine having a concentration between 3×10⁻⁶ and 3×10⁻⁴ M; the deriving step (b) includes initiating a callus culture and subculturing the callus culture at least twice; the initiating step (c) is initiating a suspension culture from the secondary or subsequent callus subculture, and further including after step (c), the step of subculturing the suspension culture at least once, before the elicitor-exposing step (d); the culture of step (c) is a differentiated liquid culture selected from embryo, root, shoot, hairy root, and teratoma; the elicitor system has at least one elicitor, each elicitor being independently selected from microorganism-derived elicitors, plant-derived elicitors, and chemically-defined elicitors (examples as above); or combinations of the above.

Embodiments of the invention which include exposure of an ungerminated seed to a DNA methylation inhibitor or exposure of a germinating seed to a DNA methylation inhibitor produce plant cell and tissue cultures which have, among other advantages, an altered phytochemical production profile that remains altered (i.e., is epigenetically stable, or does not revert) through multiple subcultures. The resulting secondary metabolites are screened for therapeutic and diagnostic applications (e.g., as anti-fungal, anti-bacterial, anti-viral, anti-inflammatory, and anti-cancer agents; or for use in clinical diagnosis, diagnostic test kits, or research purposes). Such screening employs cell-based assays, enzyme-based inhibition assays, and other methods for measuring pharmacological activity known to those in the art.

Other features and advantages of the present invention will be apparent from the following drawings and detailed description, examples, and also from the appended claims.

### BRIEF DESCRIPTION OF THE DRAWINGS

The drawings are first described.

FIG. 1 is a set of HPLC chromatograms of extracts from Buddleja davidii cell cultures subject to (A) T1, control (B) T2, (C) T3, and (D) T4 treatments.

FIG. 2 is a set of HPLC chromatograms of extracts from Calystegia sepium cell cultures subject to (A) T1, control (B) T2, (C) T3, and (D) T4 treatments.

FIG. 3 is a set of HPLC chromatograms of extracts from Lavandula sp. cell cultures subject to (A) T1, control (B) T2, (C) T3, and (D) T4 treatments.

FIG. 4 is a set of HPLC chromatograms of extracts from EC1684 and EC1692 (Eschscholtzia californica) cell cultures subject to (A) T1, EC1692 (B) T3, EC1692 (C) T1, EC1684 and (D) T3, EC1684 treatments, wherein the plant cells in (C) and (D) were derived from Eschscholtzia californica seeds pretreated with 5-azacytidine.

### DETAILED DESCRIPTION OF THE INVENTION

The invention relates to the manipulation of plant cell and tissue cultures with two types of treatment: treatment with DNA methylation inhibitors and treatment with elicitor systems. Treatment with a DNA methylation inhibitor is provided to an ungerminated seed, a germinating seed, an explant or tissue culture, or a liquid culture. Successive treatments with a DNA methylation inhibitor are also contemplated. For example, the invention encompasses a method including treating an ungerminated seed with a DNA methylation inhibitor (first treatment), germinating the treated seed, growing a callus from tissue derived from the germinated seed, inducing suspension from the callus, and treating a liquid suspension subculture with a DNA methylation inhibitor (second treatment). Whether a single treatment or successive treatments are used, ultimately, a liquid culture is derived from the DNA methylation inhibitor-treated plant cells or tissue.

DNA methylation inhibitor treatment affects the secondary metabolites produced by the treated plant cells. In part, the effect on treated plant cells is a temporary stress-induced effect. More importantly, according to the invention, the treatment also affects secondary metabolite production of subcultures derived from the treated ungerminated seed, germinating seed, explant or tissue culture, or liquid culture. This invention is based, in part, on the discovery that the effect of DNA methylation inhibitor treatment is epigenetically stable with respect to altering the expression of secondary metabolism.

According to the invention, treatment with a DNA methylation inhibitor is generally combined with treatment of the derived liquid culture with an elicitor system. Treatment with an elicitor system, i.e., elicitation, stimulates or promotes the production of phytochemicals known as secondary metabolites. Elicitation of a plant cell or tissue culture is generally performed when the plant liquid culture is established and can be grown to sufficient levels to enable the analysis of secondary metabolites. After elicitation, the phytochemicals are generally sampled or harvested for pharmacological screening, isolation, and characterization.

DNA Methylation Inhibitors

Specific examples of DNA methylation inhibitors include 5-azacytidine (5-AC), 5-aza-2′-deoxycytidine, 5-fluorocytidine, pseudoisocytidine, DL-ethionine, and 2-amino-5-ethoxy-carbonylpyrimidine-4(3H) one. As used herein, a DNA methylation inhibitor includes both a single DNA methylation inhibitor and a mixture of DNA methylation inhibitors. Exemplary protocols are found in Arfmann, et al. Z. naturforsch. (1985) 40c, 21-25; Brown et al., Theor. Appl. Genet. 78:321-328 (1989); Burn, et al., Proc. Nat'l Acad. Sci. USA 90:287-291 (1993); and Stafford et al., in MANIPULATING SECONDARY METABOLISM IN CULTURE, (ed. R. T. Robins and M.J.C. Rhodes) pp. 31-40 (1988).

Plant Species

According to some aspects of the invention, an ungerminated seed is exposed to a DNA methylation inhibitor. As used herein, a seed is the product of a fertilized ovule which can be sown and germinated to produce a seedling plant. The seed is selected from the group consisting of gymnosperms and all flowering plants, the latter being Anthophyta (formerly Angiospermae). Anthophyta contains two classes, Monocotyledonae (monocots) and Dicotyledonae (dicots) with about 241,000 species. Gymnosperms contains five extant groups including cycads, conifers and yews, with about 760 species.

Where, as in some embodiments, liquid cultures are derived directly from DNA methylation inhibitor-treated explant tissue, the explant tissue is selected from pteridophytes (e.g., clubmosses, horsetails, and ferns) and bryophytes (e.g., mosses and liverworts) in addition to the Anthophyta and gymnosperms described above. For specific species, see Thain, M., et al., The Penguin Dictionary of Biology, Penguin Books UK 9th edition, 1994, Mabberley, D. J., The Plant-Book: A Portable Dictionary of Higher Plants, Cambridge University Press 1993.

Elicitors

Specific classes of elicitors include plant-derived elicitors, microorganism-derived elicitors, and chemically-defined elicitors. First, chemically-defined elicitors include intracellular and intercellular mediators in a plant defense response, or agonists thereof, and certain inorganic salts. For example, one elicitor is methyl jasmonate, a known biological signal transducer in the plant defense pathway. Other chemically-defined elicitors include salicylic acid, glutathione, 2,6-dichloroisonicotinic acid cellulase, chitosan, nigeran, and intermediates in the peroxide cascade. Abiotic chemically-defined elicitors include silver nitrate, cupric chloride, cupric sulfate, and mercurous chloride.

Second, microorganism-derived elicitors include crude preparations or defined extracts of microorganisms (e.g., fungi, viruses, yeast, and bacteria). Specific examples of microorganisms include Candida albicans, Saccharomyces cerevisiae, Aspergillus niger, Phytophthora cryptogea, Pseudomonas syringae, and Erwinia caratovora pv. carotovora. Additional examples of bacterial elicitors are found for example in Fiedler, et al., WO89/06687, Table 2 Microorganism-derived elicitors include autoclaved whole cultures of microbial microorganisms (e.g., those recited above), and extracts, preparations, or fragments thereof.

The following are examples of microorganism-derived elicitors: yeast extract, fungal mycelia, culture broths, fungal conidial preparations, acid hydrolysates of fungal cell walls (e.g., oligosaccharides such as chitosan and other soluble carbohydrates), viral coat proteins, mycotoxins and proteins (e.g., cryptogein), bacterial toxins (e.g., syringomycin), microbial enzymes (e.g., α-1,4-endopolygalacturonic acid lyase), cellulase, xylanase (endo-(1,4)-β-xylanase), and phosphonate-treated fungal preparations. Some microorganism-derived elicitors are also chemically-defined, or available from other sources. Microorganisms may or may not be pathogenic to a chosen plant species. Extracts of varying purity are used. A representative method of preparing a microorganism-derived elicitor is described in van der Heijden, R., et al., Plant Cell Reports (1988) 7:51-54.

Elicitor Systems

According to the invention, an elicitor system is characterized by the number of elicitors, the type of elicitor(s), the sequence and duration of exposure(s), and the time period between exposures, if any. For example, one elicitor system consists of both methyl jasmonate (a chemically-defined elicitor) and an extract of Candida albicans (a microorganism-derived elicitor), wherein both elicitors are simultaneously administered once to a suspension culture.

An elicitor system includes one or more elicitors (e.g., 2, 3, 4, or more) to which a plant cell (e.g., in a culture) is exposed. Where there are two or more elicitors, the elicitors may be of the same or different elicitor class. Four examples of elicitor combinations are (i) three elicitors derived from the same microorganism; (ii) two elicitors, each derived from a different microorganism; (iii) one microorganism-derived elicitor and two chemically-defined intracellular mediators; and (iv) an inorganic salt and a bacterial toxin. Where the elicitor system is a series of elicitor treatments, the elicitors are independently selected, i.e., each treatment may include the same elicitor as another treatment, or each treatment may differ. The amounts of each elicitor in a combination may be in any non-toxic proportion, and the amount of a given elicitor may vary in each of a series of treatments. The sequence and duration of exposure to individual elicitors in an elicitor system may vary. An elicitor system can be a single brief treatment, or a series of treatments at specified times (e.g., on day 3 following 3 successive subcultures) and concentrations (e.g., 50 mg dry weight microorganism culture per liter of liquid plant cell culture). For example, another elicitor system consists of an extract of Candida albicans, administered every 48 h for the first week after a particular subculturing. The duration and frequency of treatment is dependent, in part, on the stability and metabolic fate of each elicitor, and can be modified by dilution, a change in media, or further subculture. Exemplary elicitation procedures are found in Chappell, J. and Hahlbrock, K., Nature (1984) 311:76-84; Threlfall, D. R., and Whitehead, I. M., Biochem. Soc. Trans. (1988) 16:71-75; Robbins, M. P., et al. Plant Cell Reports (1991) 10:59-62; and Kauss, H. et al., Plant Physiol. (1993) 102:459-466.

A chosen elicitor system is used to stimulate secondary metabolite production in a plant cell or tissue culture. So far, plant cultures derived from over 160 plant species, representing over 50 families, have been manipulated according to one or more methods of the invention. These include the following: Aceraceae (e.g., Acer pseudoplatanus); Aizoaceae (e.g., Mesembryanthemum crystallinum); Anacardiaceae (e.g., Rhus hirta); Apocynaceae (e.g., Mandevilla splendens, Catharanthus roseus, Rhabdadenia pohlii, Acokanthera spectabilis, and Tabernaemontana divaricata); Araliaceae (e.g., Hedera helix, Fatshedera lizei, and Hedera sp.); Betulaceae (e.g., Corylus avellana); Boraginaceae (e.g., Onosma sericeum, Anchusa azurea, and Symphytum offinicale); Caprifoliaceae (e.g., Symphoricarpos albus); Caryophyllaceae (e.g., Saponaria officinalis, Silene alba, Agrostemma gracilis, Herniaria glabra, and Dianthus barbatus); Chenopodiaceae (e.g., Chenopodium rubrum); Cistaceae (e.g., Helianthemum chamaecistus); Compositae (e.g., Carthamus tinctorius, Centaurea nigra, Echinacea purpurea, Onopordum acanthium, Conyza bonariensis, Helianthus annuus, Helichrysum italicum, Rudbeckia hirta, Artemisia annua, Artemisia absinthium, Senecio vulgaris, Aster sp., Solidago virgaurea, Anaphilus margaritacea var. yedoensis, Arctium minus, Arctium lappa, and Calendula arvensis); Convolvulaceae (e.g., Ipomea purpurea, Calystegia sepium, Ipomea batatas, and Convolvulus cneorum); Crassulaceae (e.g., Sedum spectabile); Cruciferae (e.g., Amoracia rusticana); Cucurbitaceae (e.g., Bryonia cretica); Dipsacaceae (e.g., Scabiosa columbaria); Ericaceae (e.g., Arctostaphylos densiflora); Euphorbiaceae (e.g., Euphorbia cyparissias and Ricinus communis); Geraniaceae (e.g., Geranium molle); Ginkgoaceae (e.g., Ginkgo biloba); Grossulariaceae (e.g., Ribes nigrum and Escallonia sp.); Guttiferae (e.g., Hypericum capitatum and Hypericum perforatum); Hippocastanaceae (e.g., Aesculus hippocastinum); Hydrangeaceae (e.g., Philadelphus sp.); Labiatae (e.g., Stachys sylvatica, Stachys officinalis, Teucrium fruticans, Melissa officinalis, Ocimum basilicum, Salvia officinalis, Salvia farinacea, Hyssopus officinale, Hyssopus agastache anethiodora, Prunella vulgaris, Lavandula sp., Phlomis fruticosa, and Coleus blumei); Leguminosae (e.g., Medicago sativa, Dolichos lablab, Ononis rotundifolia, Mellilotus officinalis, Indigofera tinctoria, Indigofera spinosa, Indigofera colutea, Indigofera volkensii, Trifolium repens, Acacia stricta, Wisteria sinensis, Trigonella foenum-graecum, Phaseolus vulgaris Golden Sands, Peltophorum africanum, Arachis hypogea, Glycine max, and Indigofera erecta); Linaceae (e.g., Linum usitatissimum); Loganiaceae (e.g., Buddleja davidii); Malvaceae (e.g., Gossypium hirsutum, Alcea rosea, Hibiscus mutabilis); Moraceae (e.g., Ficus religiosa and Ficus carical; Myrtaceae (e.g., Eucalyptus dalrympleana); Nyctaginaceae (e.g., Mirabilis jalapa); Nyssaceae (e.g., Camptotheca acuminata); Oleaceae (e.g., Syringa vulgaris, Jasminum x stephanense, and Ligustrum vulgare); Papaveraceae (e.g., Eschscholtzia californica); Pedaliaceae (e.g., Sesamum indicum); Phytolaccaceae (e.g., Phytolacca americana); Plantaginaceae (e.g., Plantago lanceolata); Polygonaceae (e.g., Fagopyrum esculentum, Polygonum aviculare, and Rheum palmatum); Primulaceae (e.g., Anagalis arvensis); Proteaceae (e.g., Embothrium lanceolatum); Ranunculaceae (e.g., Nigella sativa); Rosaceae (e.g., Rosa canina, Rubus tricolor, Cotoneaster horizontalis, Sorbus aucuparia, Spiraea salicifolia, Amygdalus communis, Sorbus aria, Duchesnea indica, Gardenia thunbergia, Galium aparine, Asperula orientalis, and Borreria leavis); Rutaceae (e.g., Citrus paradisi and Ruta graveolens); Saxifragaceae (e.g., Heuchera sanguinea); Scrophulariaceae (e.g., Digitalis grandiflora, Linaria purpurea, Cymbalaria muralis, Linaria dalmatica, and Linaris genistifolia); Simaroubaceae (e.g., Quassia amara); Solanaceae (e.g., Nicotiana tabacum, Nicotiana sylvestris, Nicotiana rustica, Solanum tuberosum, Solanum laciniatum, Solanum luteum, Solanum dulcemara, Lycopersicon esculentum, Lycium ferosissium, Withania somniferum, Datura sanguinea, Nicotiana glauca, Cyphomandra betacea, Hyoscyamus niger, Atropa belladonna, Schizanthus hybrid, Schizanthus x wisetonensis Star Parade, Browallia speciosa, Capsicum chinense, Capsicum frutescens, Physalis ixocarpa, and Scopolia X Petuna hybrid); Sterculiaceae (e.g., Theobroma cacao, Cola nitida, Waltheria indica, Dombeya acutangulia, and Byttnera aculeata); Umbelliferae (e.g., Daucus carota, Pimpinella anisum, Cuminum cyminum, Conopodium majus, Coriandrum sativum, Ammi majus, Pimpinella saxifraga, Anethum graveolens, and Carum petroselinum); Verbenaceae (e.g., Camara lantana); and Zingiberaceae (e.g., Brachychilum horsefieldii).

The species with one or more occurrences of improvement in T4 extracts over T1 extracts include (species/extract): Artemisia annua/E2, Anagallis arvensis/E1, Anagallis arvensis/E2, Arachis hypogea/E1, Arctium lappa/E2, Acer pseudoplatanus/E2, Bryonia cretica/E2, Buddleja davidii/E1, Conyza bonariensis/E2, Convolvulus cneorum/E1, Convolvulus cneorum/E2, Cuminum cyminum/E5, Combretum microphyllum/E1, Combretum microphyllum/E2, Conopodium majus/E2, Cola nitida/E1, Catharanthus roseus/E1, Dombeya acutangulia/E3, Dombeya acutangulia/E5, Dombeya acutangulia/E5, Digitalis grandiflora/E1, Duchesnea indica/E1, Eschscholtzia californica/E1, Eschscholtzia californica/E5, Eschscholtzia californica/E2, Embothrium lanceolatum/E1, Echinacea purpurea/E2, Escallonia sp./E1, Ficus religiosa/E1, Ginkgo biloba/E2, Hyssopus agastache anethiodora/E3, Hyssopus agastache anethiodora/E4, Hyssopus agastache anethiodora/E5, Hypericum capitatum/E1, Helichrysum italicum/E4, Hyssopus officinale/E5, Lavandula sp./E2, Mesembryanthemum crystallinum/E1, Nicotiana sylvestris/E2, Ocimum basilicum/E1, Symphoricarpos albus/E1, Scabiosa columbaria/E1, Scabiosa columbaria/E5, Salvia officinalis/E2, Stachys sylvatica/E3, Stachys sylvatica/E5, Spiraea salicifolia/E5, Syringa vulgaris/E1, Senecio vulgaris/E1, Senecio vulgaris/E2, and Theobroma cacao/E1. These represent preferred families and preferred species.

Improvement under the above conditions does not preclude improvement under other conditions encompassed by the methods of the invention such as other elicitor systems and different DNA methylation inhibitors. Similarly, a particular extract may test positively in one screening assay but not in another. Thus, cultures of the following species have also been successfully manipulated according to the invention as measured by, e.g., HPLC profile or screening assay: Atropa belladonna, Amygdalus communis, Agrostemma gracilis, Anethum graveolens, Aesculus hippocastanum, Ammi majus, Anaphilus margaritacea, Arctostaphylos densiflora, Asperula orientalis, Alcea rosea, Armoracia rusticana, Acokanthera spectabilis, Byttneria aculeata, Browallia speciosa, Calendula arvensis, Corylus avellana, Cyphomandra betacea, Coleus blumei, Capsicum chinense, Capsicum frutescens, Cotoneaster horizontalis, Camara lantana, Cymbalaria muralis, Combretum microphyllum, Centaurea nigra, Carum petroselinum, Citrus paradisi, Chenopodium rubrum, Calystegia sepium, Coriandrum satvium, Carthamus tinctorius, Dianthus barbatus, Daucus carota, Dolichos lablab, Euphorbia cyparissias, Eucalyptus dalrympleana, Ficus carica, Fagopyrum esculentum, Fatshedera lizei, Galium aparine, Gossypium hirsutum, Glycine max, Geranium molle, Gardenia thunbergia, Helianthus annuus, Helianthemum chamaecistum, Hedera helix, Hibiscus mutabilis, Hyoscyamus niger, Hypericum perforatum, Heuchera sanguinea, Hedera sp., Ipomea batatas, Indigofera colutea, Ipomea purpurea, Indigofera spinosa, Indigofera tinctoria, Indigofera volkensii, Jasminum x Stephanense, Linaria dalmatica, Lycopersicon esculentum, Lycium ferocissium, Linaria genistifolia, Linum usitatissimum, Ligustrum vulgare, Mirabilis jalapa, Melilotis officinalis, Medicago sativa, Mandevilla splendens, Nicotiana glauca, Nicotiana rustica, Nigella sativa, Nicotiana tabacum, Onopordum acanthium, Ononis rotundifolia, Onosma sericeum, Polygonum aviculare, Pimpinella anisum, Phytolacca americiana, Phlomis fruticosa, Physalis ixocarpa, Plantago lanceolata, Philadelphus sp., Pimpinella saxifraga, Phaseolus vulgaris Golden, Prunella vulgaris, Quassia amara, Rosa canina, Ruta graveolens, Rudbeckia hirta, Rhus hirta, Ribes nigrum, Rhabdadenia pohlii, Rheum palmatum, Ribes rubrum, Sorbus aria, Silene alba, Simmondsia chinensis, Solanum dulcemara, Salvia farinacea, Schizanthus hybrid, Sesamum indicum, Solanum laciniatum, Solanum luteum, Stachys officinalis Rosea, Symphytum officinale, Scopolia x Petunia hybrid, Sedum spectabile, Solanum tuberosum, Solidaga vigaurea, Schizanthus x wisetonensis, Tabernaemontana divaricata, Teucrium fruticans, Trigonella foenum-graecum, Trifolium repens, Waltheria indica, Wisteria sinsensis, and Withania somniferum.

More preferred species include Stachys sylvatica, E. californica, Helianthus annuus, Senecio vulgaris, Prunella vulgaris, Conopodium majus, Syringa vulgaris, Scabiosa columbaria, Nicotiana rustica, Ligustrum vulgare, Gossypium hirsutum, Onosma sericeum, Calystegia sepium, Convolvulus cneorum, Buddleja davidii, Phlomis fruticosa, Polygonum aviculare, Arachis hypogea, Artemisia annua, Salvia officinalis, Alcea rosea, Hibiscus mutabilis, Mirabilis jalapa, Dombeya acutangulia, Acer pseudoplatanus, Hyssopus officinale, and Ficus religiosa. More preferred families include the families of the species named in this paragraph.

Exposing an ungerminated seed to a DNA methylation inhibitor may be accomplished by any method, including soaking, imbibing, spraying, injection, or controlled release technologies. It is believed that the effect of exposure to DNA methylation inhibitor depends upon factors including concentration, duration, method of exposure, and the presence and proportion of the dividing plant cell population. Soaking is a preferred method of seed exposure. The soaking concentration of DNA methylation inhibitors is between 1×10⁻⁷ and 5×10⁻³ M in sterile water, for example, between 1×10⁻⁶ and 6×10⁻⁴ M, and between 3×10⁻⁶ and 3×10⁻⁴ M. Concentrations of 10⁻³ M or more may be toxic. In addition to sterile water, other biocompatible fluids such as buffers and growth media solutions may be used. The duration of seed exposure is between 1 h and 7 days (e.g. 1 h-72 h, and 12 h-48 h), depending on factors such as the selection and concentration of the DNA methylation inhibitor. Some pretreatment of the seed, such as scarifying a legume seed to facilitate imbibition and germination, may be necessary. In addition, physiological dormancy of an ungerminated but imbibed seed may require pre-treatment (e.g., cold temperature treatment at 2-10° C. for several days or weeks, or hormone treatment) to overcome dormancy before a DNA methylation inhibitor is applied. In some embodiments, where such pre-treatment is lengthy, the DNA methylation inhibitor is supplied during germination and thus after imbibition. Generally, seeds are pretreated in the dark.

Another aspect of the invention relates to exposing a germinating seed to a DNA methylation inhibitor. Such exposure includes any method described above in seed treatment, and also includes adding a DNA methylation inhibitor to the germination media directly, in solution, in a liquid or solid medium, by spray application, or by a controlled-release technology. The germination concentration of a DNA methylation inhibitor is between 1×10⁻⁷ and 1×10⁻⁷ and 1×10⁻² M (e.g., between 1×10^{31 6} and 6×10⁻⁴ M or between 3×10⁻⁵ and 3×10⁻⁴ M) in sterile water or other physiologically-acceptable medium. The duration of germination treatment is between 12 h and 7 days, preferably between 2 days and 6.5 days, and more preferably between 3 and 6 days. Determination of germination treatment relies on the same factors mentioned above in seed exposure.

After germination, plant tissue is cultured to induce callus formation. A callus is a mass of undifferentiated plant cells. In some embodiments, an initial callus culture is subcultured at least once (e.g., at least 2, 3, 4, or 5 times). A callus (or alternatively other explant tissue such as sterilized stem nodes, leaf discs, or seedlings) can be used to initiate liquid plant cell and tissue cultures. A liquid culture is a differentiated culture, or an undifferentiated (e.g., suspension) culture. Examples of differentiated cultures in this context include root, shoot, or embryo. Hairy root, teratoma, root, shoot, and embryo cultures can also be derived directly from explant material without going through a discrete callus stage.

Furthermore, root cultures can be derived from plant tissue by genetic transformation with Agrobacterium rhizogenes. Infection of host plant tissue such as sterile seedlings or leaf discs with A. rhizogenes induces the formation of roots at the site of infection. The infecting bacteria can be removed by treatment of the transformed tissue with antibiotics such as carbenicillin or cefotaxime. In some species, particularly of the family Solanaceae, these root cultures are often fast-growing, and are maintained indefinitely, e.g., by transfer of excised root tips to fresh growth medium.

Another aspect of the invention relates to exposing a liquid plant cell culture or tissue to a DNA methylation inhibitor such as 5-AC. In some embodiments, the suspension culture is subcultured at least once (e.g., at least 2, 3, 4, 5, 10, or 15 times) before treatment with a DNA methylation inhibitor. Suspension exposure includes any method described above in seed treatment, and adding the DNA methylation inhibitor to the suspension media directly, in solution, or by a controlled-release technology. The suspension concentration of the DNA methylation inhibitor is between 1×10⁻⁷ and 1×10⁻² M in sterile water or other physiologically-acceptable medium, (e.g., between 1×10⁻⁶ and 6×10⁻⁴ M, or between 3×10⁻⁶ and 3×10⁻⁴ M). The duration of suspension treatment is between 2 h and 7 days, preferably between 2 h and 5 days, and more preferably between 6 h and 2 days.

It is desirable to expose the suspension culture to a DNA methylation inhibitor during the peak of mitotic activity, to affect the largest proportion of dividing cells. This peak usually occurs between 2 and 4 days after initiating a subculture. Although the timing of the peak can vary among species, the peak is either known or easily determined by persons skilled in the art for any given species.

To avoid evaluating phytochemicals produced from mere cellular stress due to a DNA methylation inhibitor such as 5-AC, a suspension culture which has been exposed to a DNA methylation inhibitor is subcultured at least once (e.g., at least 2, 3, 5, or 10 times) before exposure to an elicitor system. While repeated application of a DNA methylation inhibitor is possible, it is believed that a single application timed to affect a large dividing population is most effective, and therefore is preferable. Moreover, 5-AC is not stable in physiological solutions over long periods of time.

In some embodiments, after the step of subculturing a DNA methylation inhibitor-exposed liquid culture (e.g., suspension, root, shoot, or embryo culture), there is a further step, such as: storing the subculture at a temperature between 4° C. and 20° C.; storing the subculture at a temperature between −80° C. and −10° C.; storing the subculture at a cryogenic temperature between −196° C. and −170° C.; exposing the subculture to a DNA methylation inhibitor before exposing the subculture (or a subsequent subculture derived therefrom) to an elicitor system wherein the exposure of the subculture to a DNA methylation inhibitor is performed between 1 and 10 times (e.g., between 2 and 5 times, or 1, 2 or 3 times); and combinations thereof. See Example 9 below, and, e.g., Grout, B., et al., TIBTECH (October, 1990) 293-297, Diettrich, B., et al., J. Plant Physiol. (1986) 126:63-73, Bajaj, Y.P.S., BIOTECHNOLOGY IN AGRICULTURE AND FORESTRY, (1984) Vol. 4, Chapter I.8, 169-, Chen, T.H.H., et al., Plant Physiol. (1984) 75:726-731, and Butenko, R. G. et al., Plant Sci. Lett., (1984) 33:285-292.

One embodiment of the invention is the method of affecting secondary metabolite production in a plant culture, comprising: obtaining a seed; exposing the seed to a DNA methylation inhibitor; initiating a culture from tissue derived from the DNA methylation inhibitor-exposed seed; exposing a subculture derived from the initiated culture to a DNA methylation inhibitor; exposing a subculture derived from the DNA methylation inhibitor-exposed culture to an elicitor system; and maintaining the elicitor-exposed subculture. In certain embodiments, the elicitor system is (a) methyl jasmonate or (b) a simultaneously-administered combination of methyl jasmonate and a microorganism-derived elicitor, such as autoclaved Candida albicans. In one embodiment, a liquid suspension culture is induced directly from a seed which may or may not be pre-treated with a DNA methylation inhibitor.

Phytochemical production is optimized in part by adjusting the amount of nutrients normally present in growth media. Such substances include auxins, sucrose, nitrate, and phosphate. For example, in one embodiment, the sucrose concentration was increased from 2% to 5%, and plant hormone 2,4-dichlorophenoxyacetic acid (2,4-D) was omitted. A person of skill in the art will easily be able to determine what culture media are appropriate. Exemplary growth media are commercially available, e.g., from Sigma Chemical Company, St. Louis, Mo., and Gibco BRL Life Technologies, Grand Island, N.Y. Typical growth media to support growth of undifferentiated cultures in solid or liquid form are Gamborg's B5 medium (Exp. Cell Res., 50:151 (1968) with the inclusion of phytohormones 2,4-D or a-naphthaleneacetic acid (NAA) at between 0.1-5 mg/L and kinetin at between 0.1-2 mg/L. Growth media developed for orchid seedling multiplication include the formulation of Vacin and Went, Botanical Gazette, 110:605 (1949).

Regarding another aspect of phytochemical production, there are methods of driving the synthetic equilibrium in the desired direction. These equilibrium-based methods include (i) adding precursors of secondary metabolites to the media and (ii) sequestering the desired metabolite. In aqueous growth media, relatively nonpolar metabolites selectively and reversibly bind to nonionic, polymeric absorbent resins such as XAD-7 (Sigma Chemical Co.).

In addition, immobilization of plant cultures affects phytochemical production. Immobilization of plant cultures in calcium alginate beads or on other inert matrices can increase the rate of phytochemical production, and alter the equilibrium between intracellular and ext:racellular metabolites. Finally, lowering ambient temperature of the liquid culture (e.g., to 20° C. or 15° C.) tends to slow culture growth and favor secondary metabolite production. Any of the above techniques can be combined with the methods of the present invention.

Stimulation and alteration of secondary metabolite production are measurable by several methods known to those in the art. For example, organic solvent extracts can be analyzed by HPLC to determine qualitatively and quantitatively whether novel phytochemicals, or increased levels of naturally-occurring phytochemicals, have been produced. Exemplary extractions include the following two extraction series. In the first series, dry biomass was extracted with 1:1 methylene chloride:methanol (E1 extraction), then the biomass was extracted with water (E2 extraction). In the second series, the biomass was first extracted with water. This aqueous extract was run through a reverse-phase resin column (the aqueous eluent being an E4 extraction). The reverse-phase column was eluted with acetonitrile (E3 extraction). The organic layer resulting from a further extraction of the aqueous E4 extraction with 1:1 methylene chloride:methanol produced an E5 extraction. Before further characterization, extractions were generally concentrated. A typical HPLC analysis is described in Example 1 below. The chromatograms reproduced hereinhave a cleaner, flatter baseline than some of the chromatograms we have obtained, some of which (not shown) have a rolling or curved baseline as a result of background impurities not uncommon in plant extracts. Even with the latter chromatograms with rolling baselines, however, the qualitative differences resulting from extraction and treatment are apparent.

In addition to HPLC analysis, the extracts (or compounds isolated therefrom) can be screened for pharmacological activity. Examples of pharmacological activity include anti-viral, anti-cancer, anti-fungal, anti-bacterial, and anti-inflammatory activities. Pharmacological activity also includes immunological activity, cardiovascular activity, and agonist or antagonist activity with respect to neurotransmitters such as acetylcholine, serotonin, and glutamate. Specific examples of pharmacological activity assays include those which measure inhibition of the following: herpes simplex virus type-2, hepatitis C virus ATPase, HIV reverse transcriptase, HIV protease, C. albicans growth (e.g., 24433 strain and 90028 strain), chitin synthase, glucan synthase, Staphlococcus aureus growth, human Cytomegalovirus (CMV) protease, HIV integrase, and amyloid precursor protein production. These assays include both enzyme- and cell-based assays adapted from the literature. See, e.g., Suzich, J. A., et al., J. Virology (1993) 67:6152-6158, run in 96-well plate format for high throughput with a reaction volume reduced from 1 mL to 100 μL (hepatitis C virus ATPase); August, E. M., et al., Biochem. Pharmacol. (1993) 45:223-230 (HIV reverse transcriptase, DNA polymerase alpha, and CMV polymerase) run in 96-well plate format with use of a 96-well harvester; Elion, G. B., et al., Proc. Nat'l. Acad. Sci. USA (1977) 74:5716-5720 (herpes simplex virus DNA polymerase) run in 96-well plate format with use of a 96-well harvester; and Roehm, N. W., et al., J. Immuno. Meth. (1991) 142:257-265 (Vero, U937, and antifungal assays (e.g., C. albicans growth) run in 96-well plate format. See Examples 2, and 4-8 below. The extracts can also be screened for inhibitory activity of additional enzymes, such as Bacillus subtilis DNA polymerase III.

Certain extracts have been screened for cell toxicity (Vero cell lines, U937 (human monocytic cell line). Specificity was evaluated by pairing a viral enzyme with a corresponding host organism enzyme (e.g., CMV DNA polymerase paired with DNA polymerase from calf thymus tissue as a control; proteases paired with pepsin as a control).

In one embodiment, plant cultures from each species are treated with one of the T2, T3, and T4 treatments, plus control (T1). The control group, grown under normal conditions in the absence of both elicitors and any DNA methylation inhibitors, was designated as T1. The T2 group was exposed to the elicitor methyl jasmonate. The T3 group was exposed to a elicitor system consisting of methyl jasmonate and autoclaved Candida albicans. The T4 group was first exposed to 5-azacytidine, a DNA methylation inhibitor; subcultured four times; and then exposed to an elicitor system consisting of methyl jasmonate and autoclaved Candida albicans.

In general, multiple extracts (e.g., E1-E5) from cultures subject to these four conditions (T1-T4) were screened in over 10 different assays, although not every combination of plant species and treatment has been tested in every assay. In addition, in some cases, multiple cultures of the same species were prepared.

The claimed methods are judged to have successfully affected the secondary metabolite production or secondary metabolite production profile of a plant cell species when an extract derived from a culture subjected to treatment and elicitation (whether or not elicitation is a step included in the particular method) has one or more particular properties. Examples of such properties include: (1) showing improved activity in at least one assay when compared with the corresponding extract derived from an appropriate control (e.g., T1); (2) containing relatively increased concentrations of a naturally-occuring product (enhanced or elevated levels of production, e.g., as shown by HPLC or other methods known to those in the art); (3) containing products not detectable in untreated cultures; and (4) containing a product that is structurally related (an analog) to a known secondary metabolite, wherein the secondary metabolite analog was obtained by adding a metabolic precursor (e.g., a primary substrate or an intermediate) to a growth medium (e.g., suspension culture medium). The primary substrate or intermediate can be a natural product, a semisynthetic product, or a wholly synthetic analog (for example, fluorinated secondary metabolites are produced by adding fluorinated metabolic precursors). Other pairs of metabolic product and precursor include alkaloids and amino acids (e.g., indole alkaloids and tryptophan), and terpenoids and either acetate or isopentenyl pyrophosphate.

Improved activity as used herein includes at least one of the following: an increased percent inhibition (in an inhibition assay) and increased specificity (e.g., specificity for pathogenic enzyme over host enzyme). An increased percent inhibition implies a lower IC₅₀, which may also be used as an indicator of improved activity. Of course, a given combination of species, treatment, and extract may result in improved activity in at least 2 or more assays (e.g., at least 3, or 4 or more assays). An extract or compound with increased specificity demonstrates one or more of the following: preferential inhibition of a pathogenic (e.g., viral, bacterial, or fungal) enzyme over a corresponding or similar enzyme in the host cell or tissue; preferential inhibition of one pathogenic enzyme over another pathogenic enzyme; and preferential inhibition of the growth of a pathogen over the growth of host cell or tissue.

Without further elaboration, it is believed that, based on the description herein, the present invention can be utilized to its fullest extent. All publications and patents mentioned herein are hereby incorporated by reference. The following specific exampls are to be construed as merely illustrative, and not limitative of the remainder of the disclosure.

### EXAMPLES

### Example 1

HPLC Analysis of Altered Secondary Metabolite Production

Twelve cell lines were selected: Buddleja davidii (Loganiaceae), Calystegia sepium (Convolvulaceae), Lavendula sp. (Labiatae), Ocimum basilicum (Labiatae), Ribes nigrum (Grossulariaceae), Scopolia x Petunia (Solanaceae), Solanum tuberosum (Solanaceae), Theobroma cacao (Sterculiaceae), Trigonella foenum-graecum (Leguminosae) (two batches), Fagopyrum esculentum (Polygonaceae), and Helianthus annuus (Compositae).

Each cell line was grown under 4 different conditions (T1-T4) as described above. More specifically, the T2 treatment involved transferring cultures by volume subculture to a production medium. Seven days after subculture, methyl jasmonate was added at a final concentration of 250 μM. Cultures were harvested 3-5 days following elicitation. The T3 treatment was identical to T2, except that a C. albicans preparation was added at a final concentration of 50 mg/L at the same time as the methyl jasmonate. In the T4 treatment, the subculture was first exposed to 5-azacytidine at a final concentration of 3×10⁻⁵ M on the third day after subculture. After four subcultures, a combination of methyl jasmonate and a C. albicans-derived preparation (as above in T3) was added.

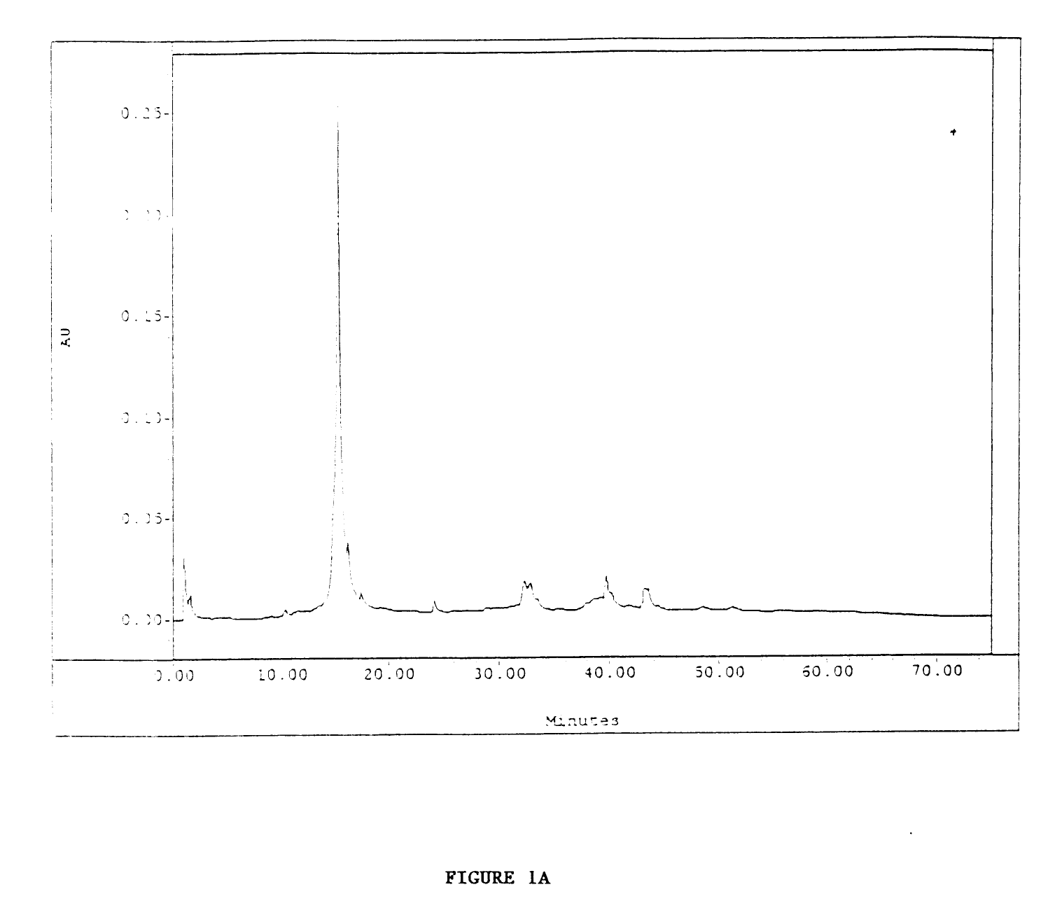

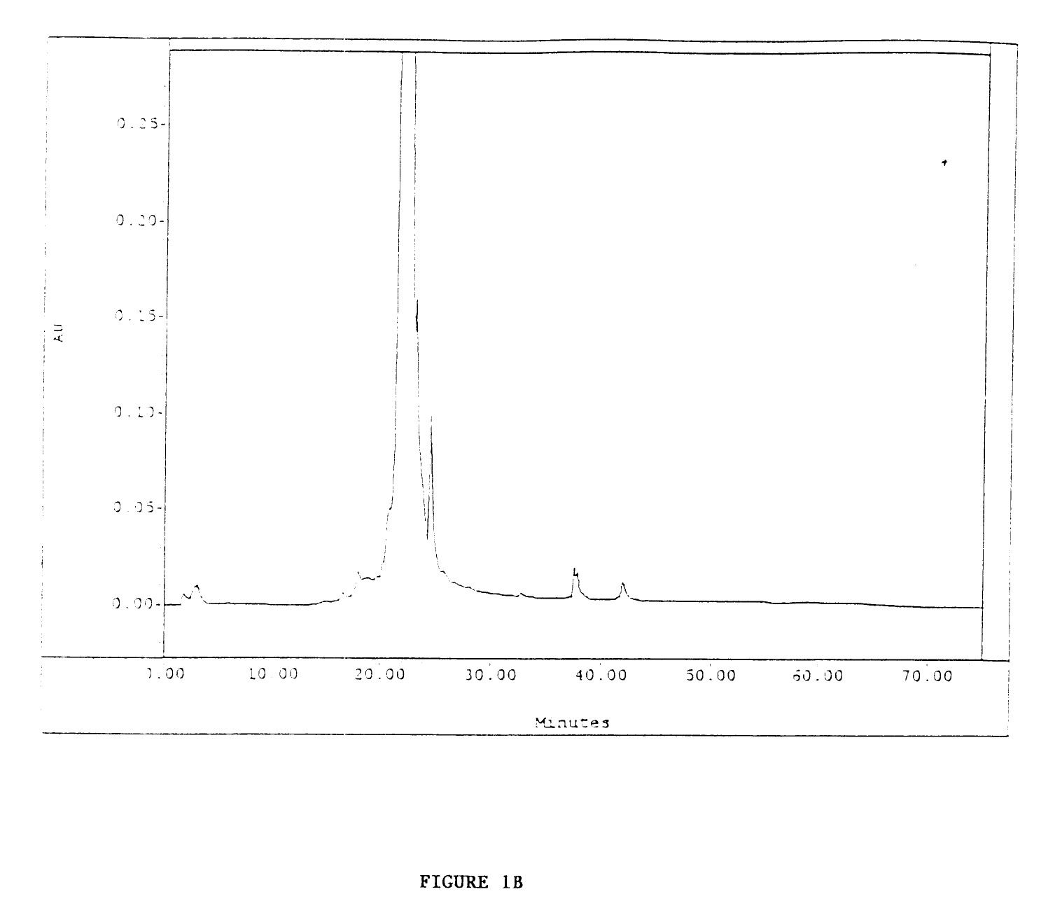

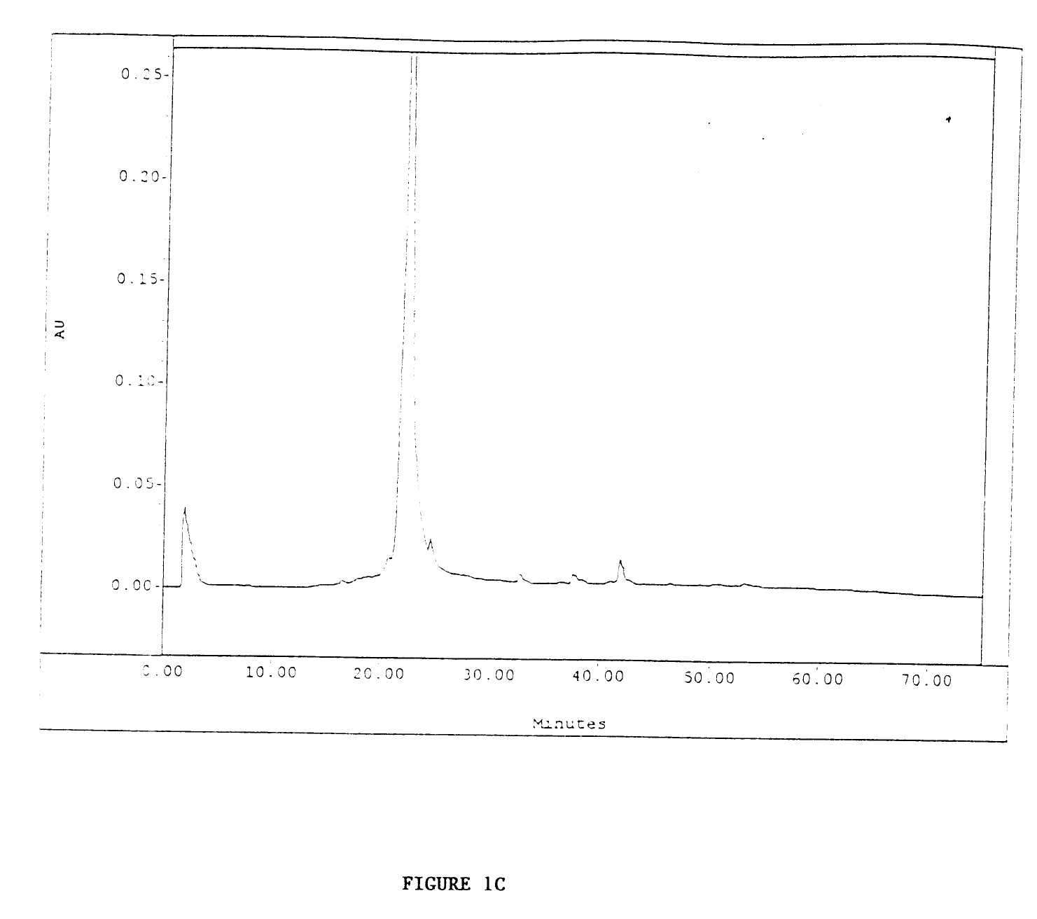

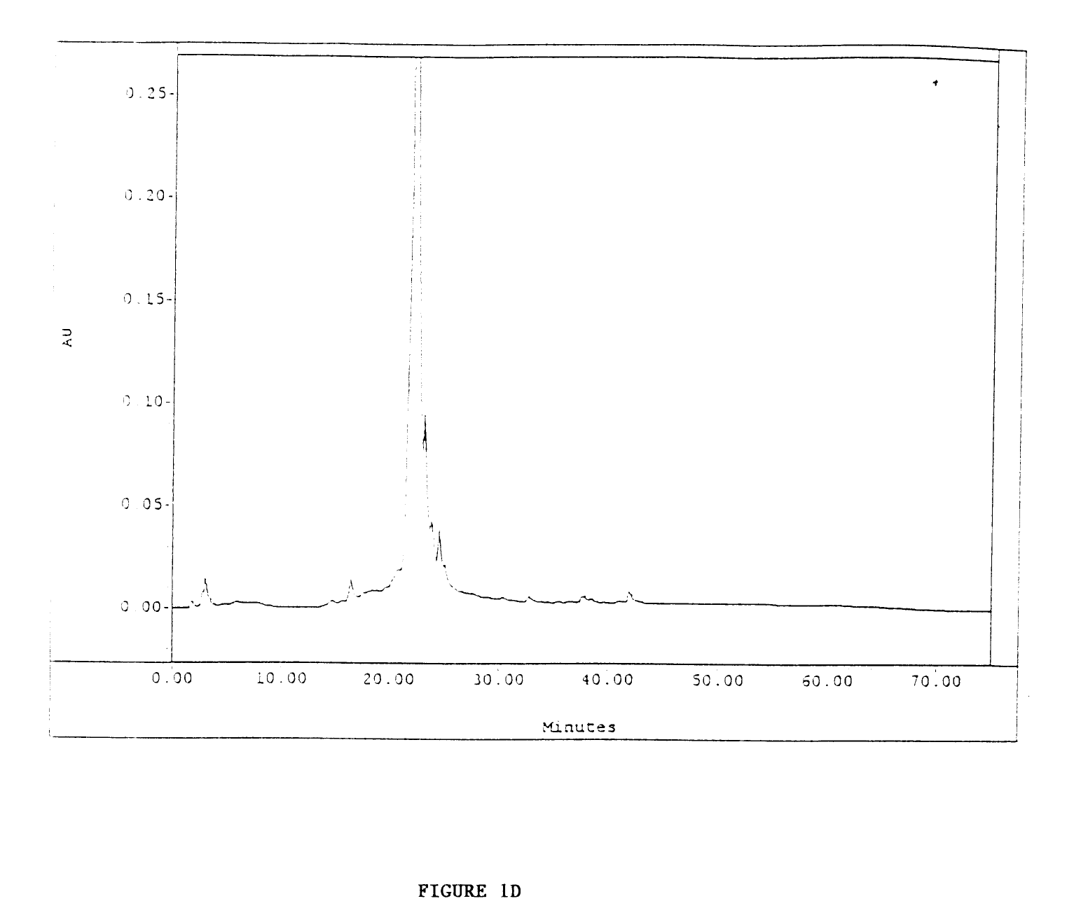

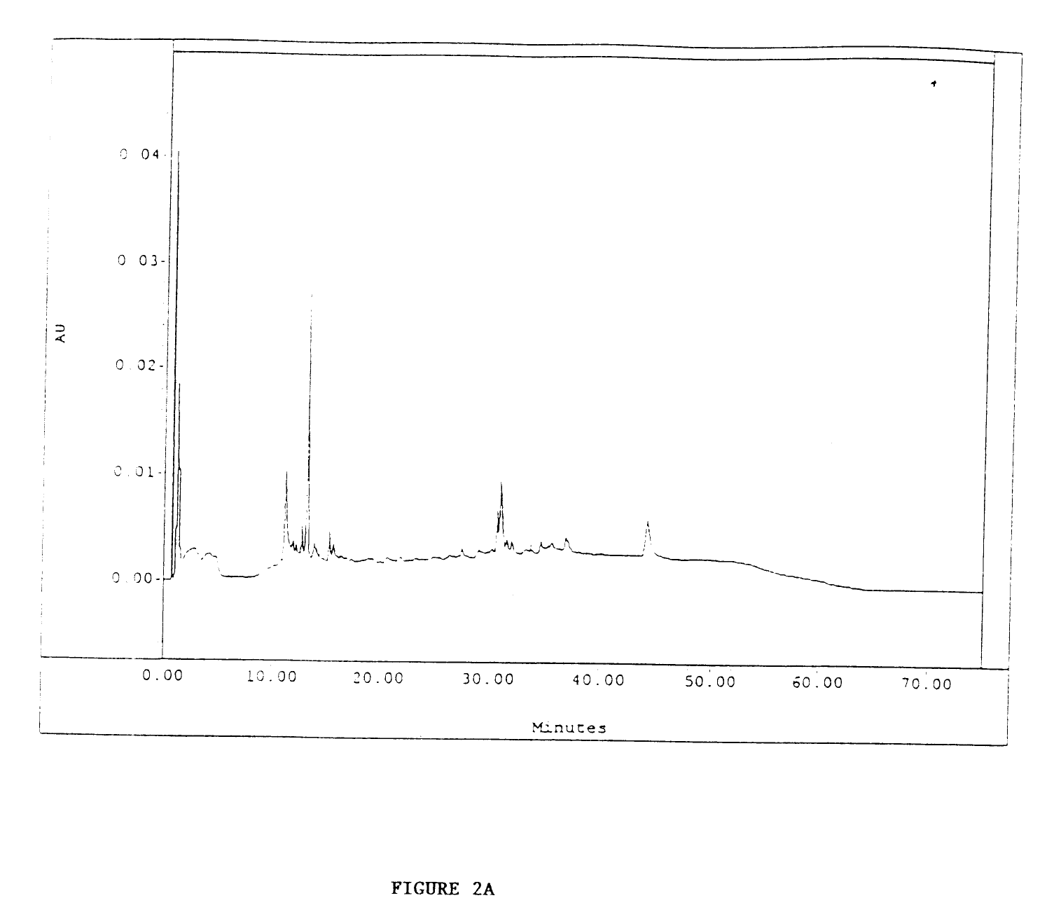

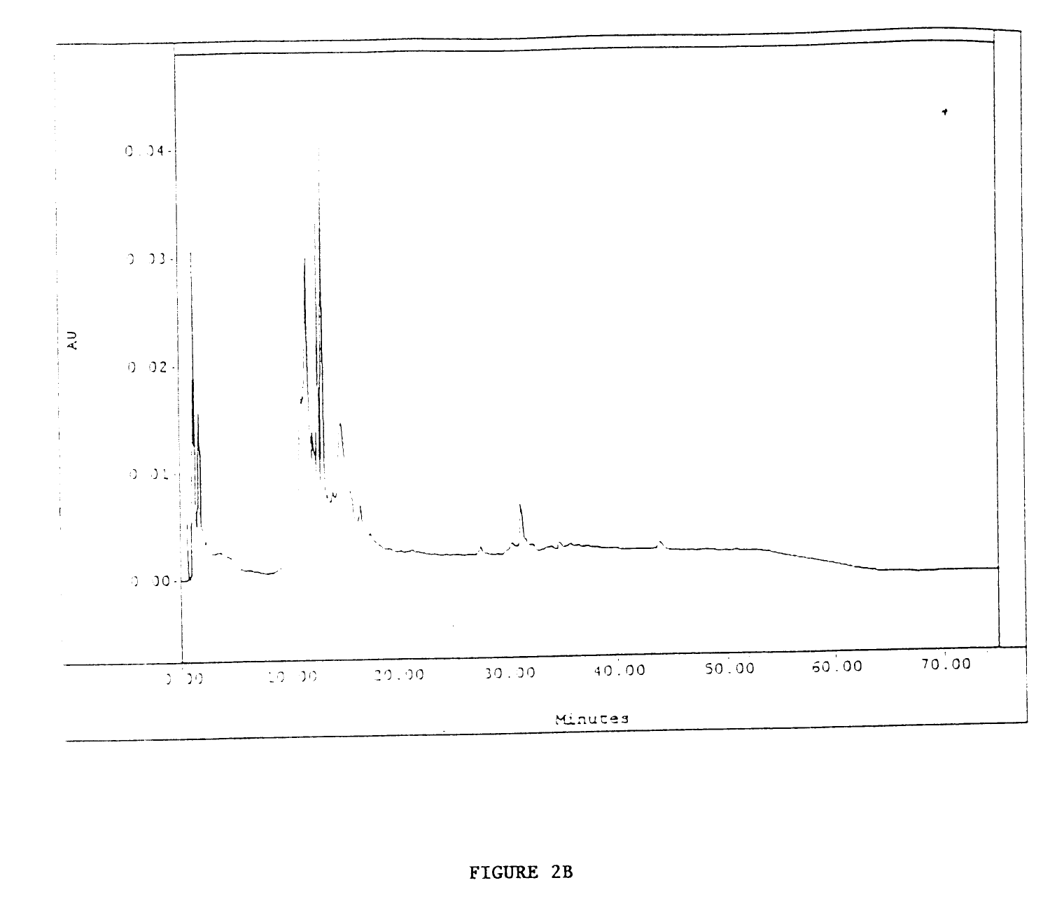

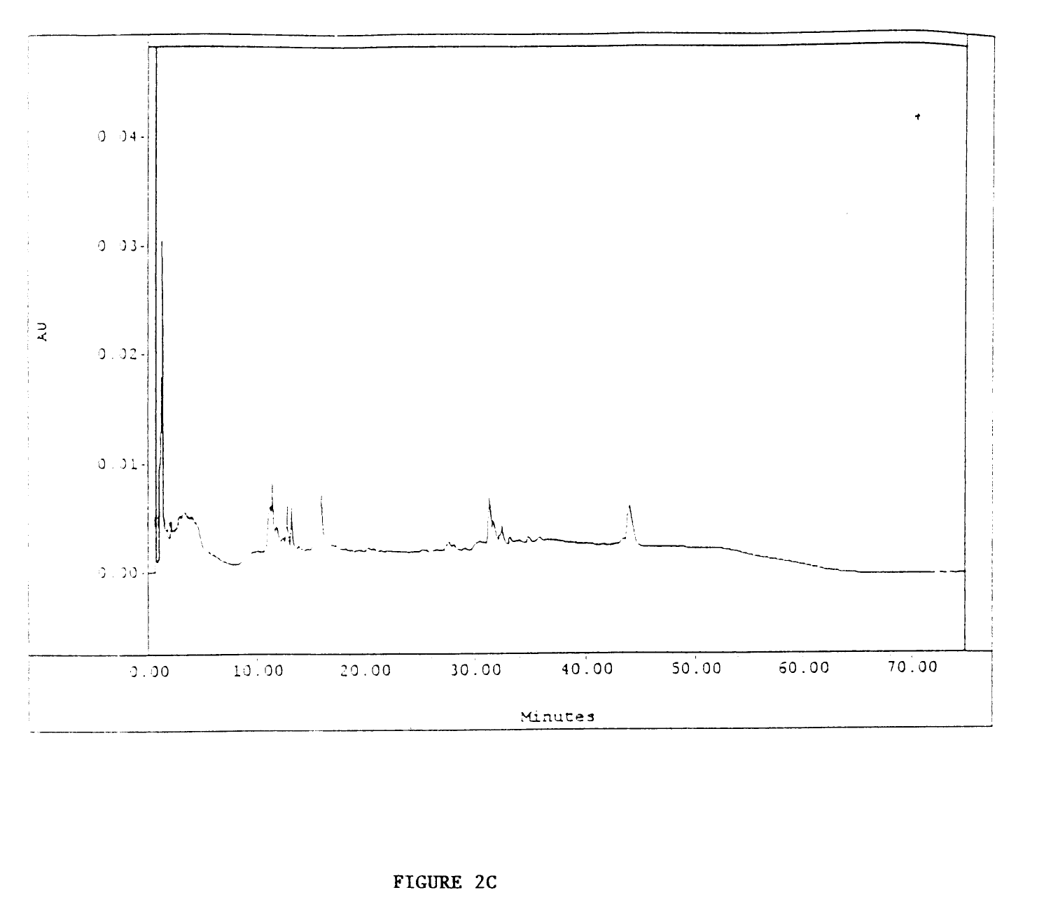

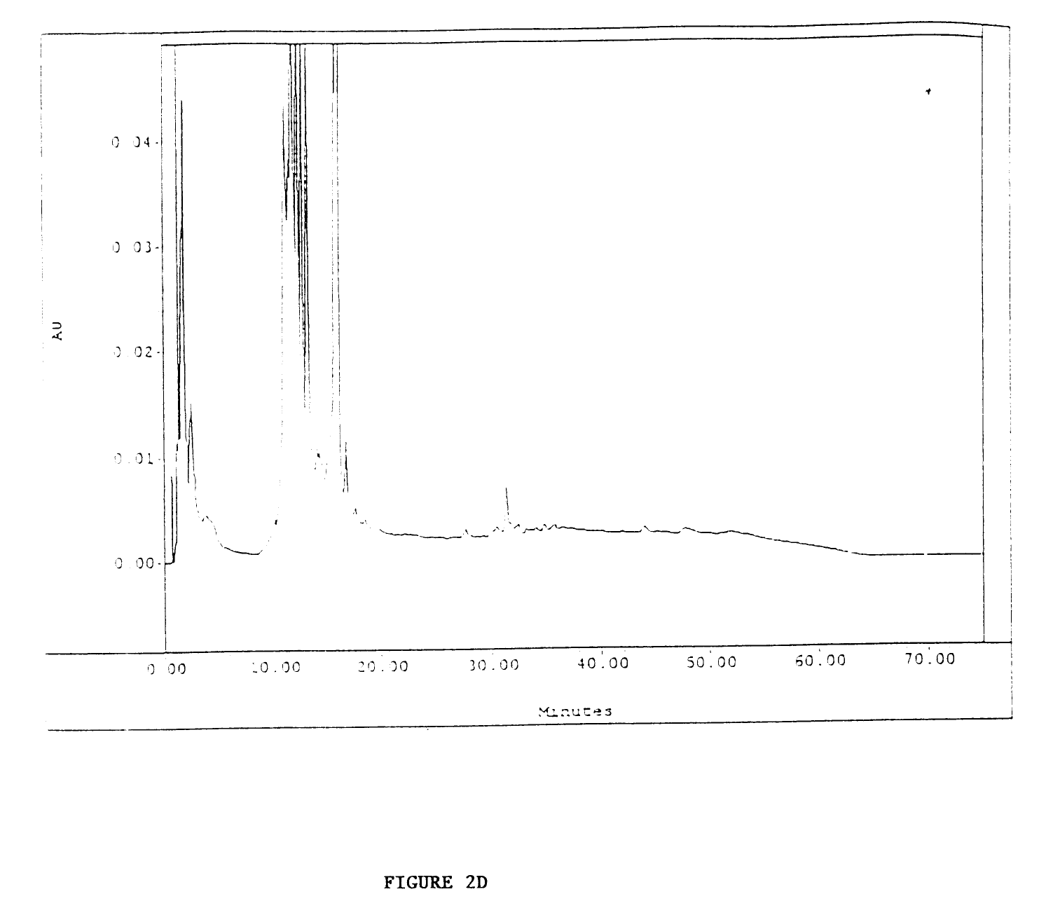

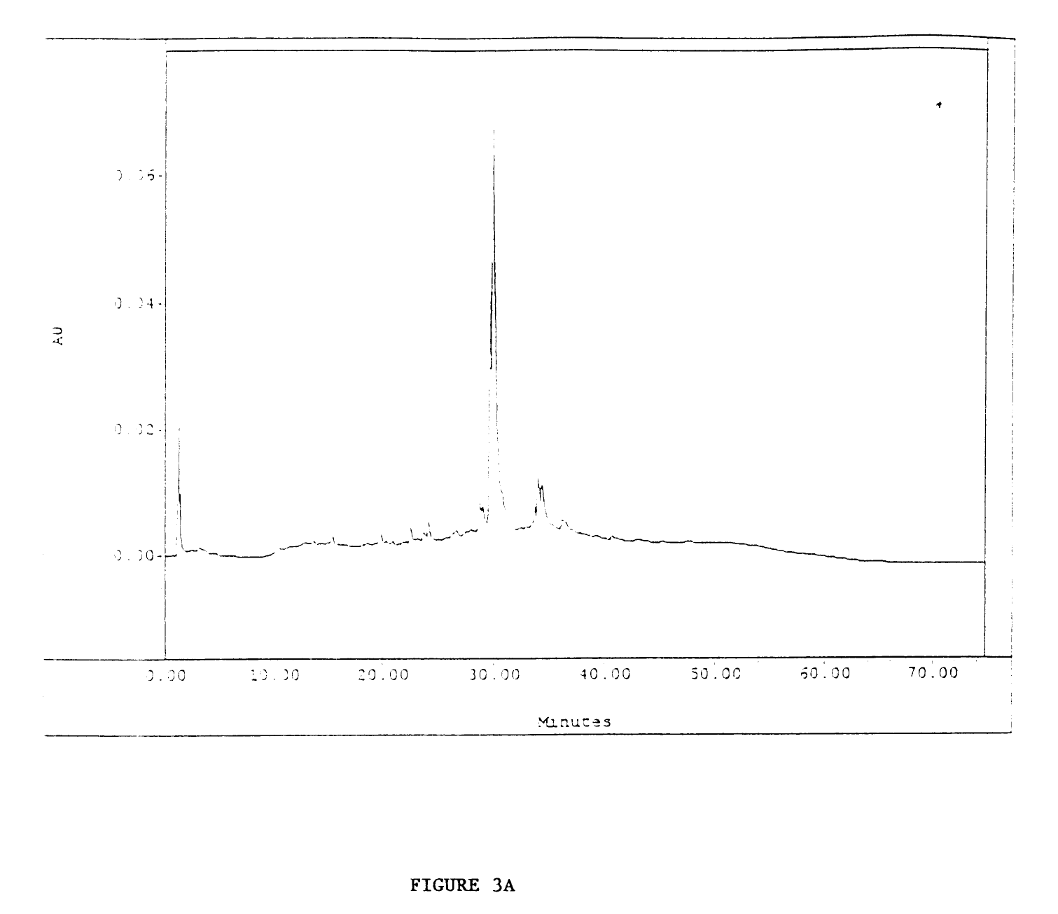

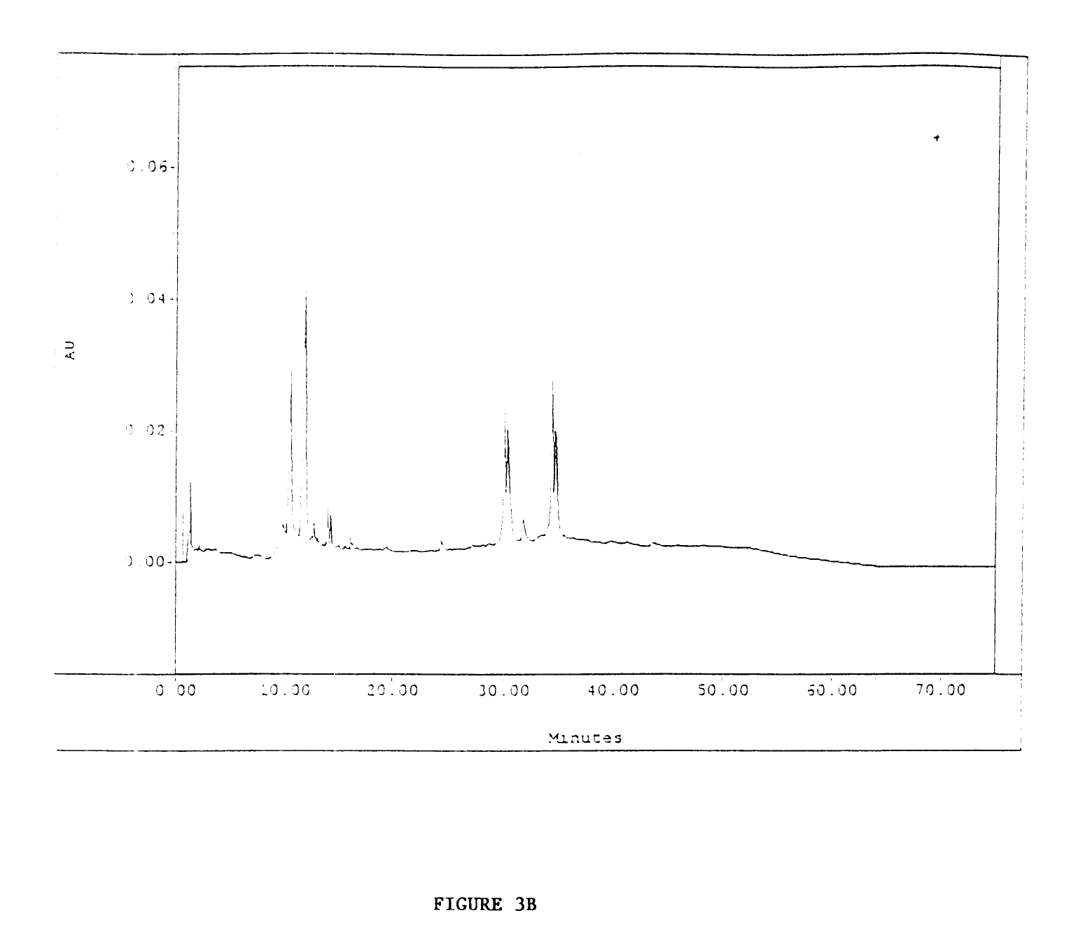

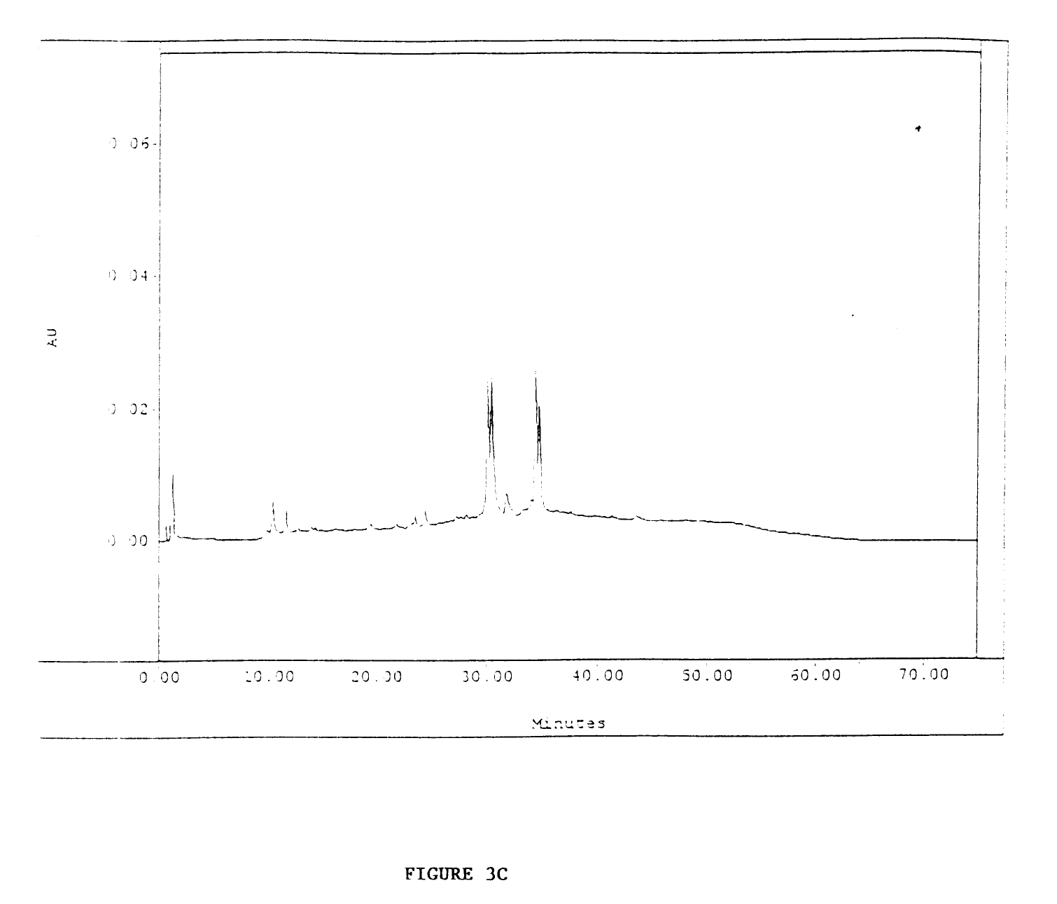

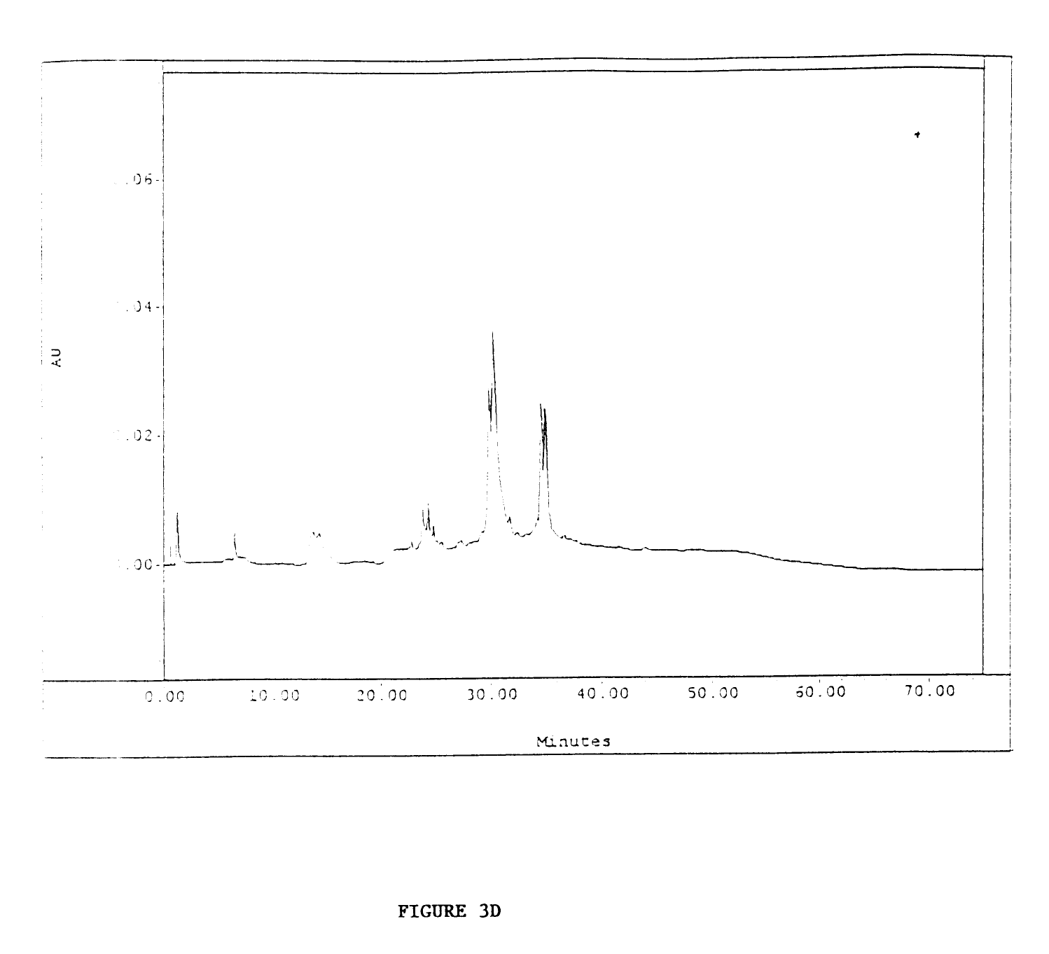

The freeze-dried cell biomasses were extracted with methylene chloride/methanol and analyzed by HPLC equipped with a 280 nm UV detector. The elution conditions were standardized: 10 mg/mL extracts, 20 μL injection volume, Nova-Pak C-18 (60 Å, 4 μm, 3.9×150 mm) column, and a 280 nm UV detector. The solvent gradient was as follows (time in minutes, % water, % methanol, % acetonitrile): (0, 100, 0, 0); (30, 10, 10, 80); (45, 10, 10, 80); (55, 100, 0, 0); and (75, 100, 0, 0). Compared with the HPLC profiles of the T1 controls, the profiles of the T2-T4 groups clearly indicated altered phytochemical production, based on the size, number, and location of the peaks (See FIGS. 1-3).

### Example 2

Inhibition of Viral Infection

Extracts prepared from Syringa vulgaris and Helianthus annuus cultures were screened for inhibitory activity in a herpes simplex virus type-2 growth inhibition assay. In a 96-well format, mammalian vero cells infected with HSV-2 (strain MS) were contacted with 100 μg/mL of extract 1 h after virus adsorbtion. Virus, cells and extract were incubated for 18 h at 37° C. in 5% CO₂ and then fixed with formalin. The extent of virus propagation was evaluated by measuring the expression of virus-specific cell-surface antigens using an ELISA format with polyclonal anti-HSV-2 antisera (DAKO). Viral infection was quantitated by comparing the O. D. values of viral-infected to uninfected controls on each plate. These controls were also tested for non-specific antibody binding by using non-immune polyclonal antisera. In addition, a positive control antiviral agent (either acyclovir or foscarnet) was included in each assay plate.

The percent inhibition of virus antigen expression by test samples (extract or positive control antiviral agent) was determined by the formula 100-[(O. D. of test sample÷O. D. infected control)×100] where the O. D. of uninfected control was subtracted from both test sample and infected control. For both cell lines, extracts from the T2-T4 groups generally showed increased activity when compared with extracts from T1 (control). The generally enhanced inhibitory activity demonstrates the effect of the combination, (a) pre-elicitation treatment with DNA methylation inhibitors and (b) treatment with an elicitor system, on secondary metabolite production in plant cells.

### Example 3

Seed Pretreatment

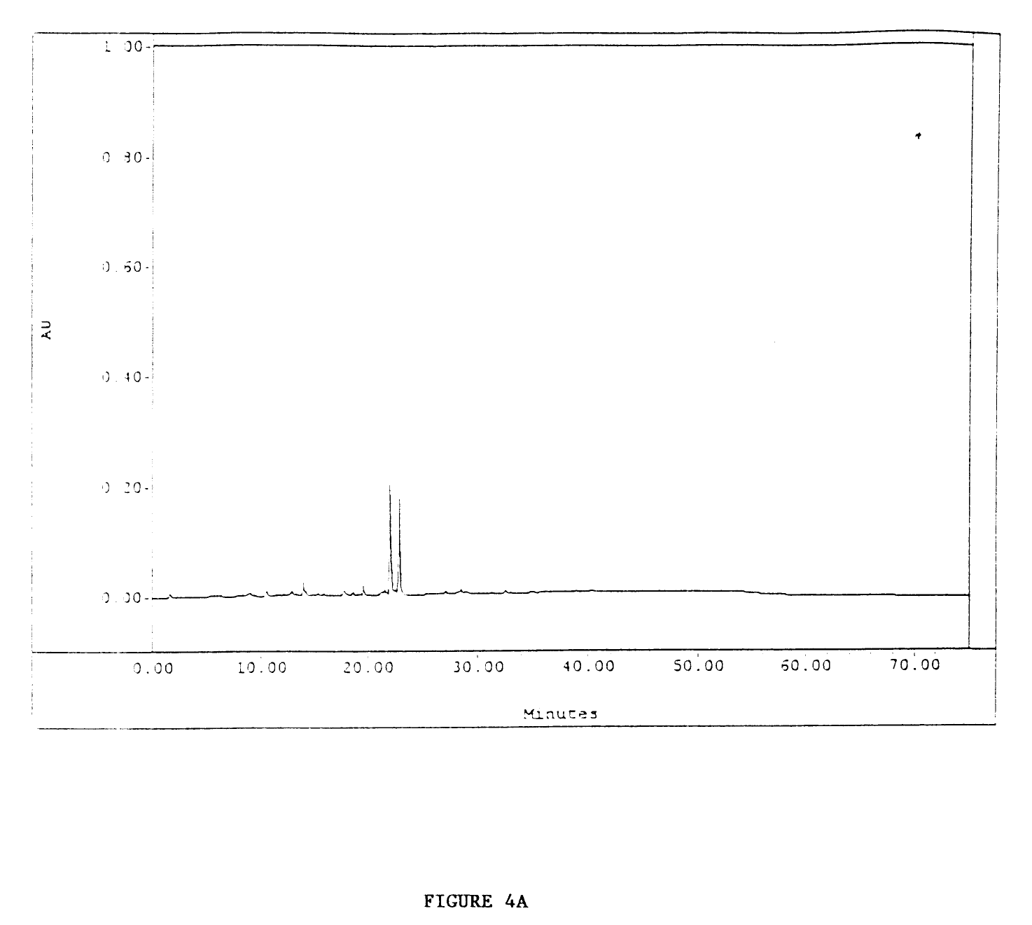

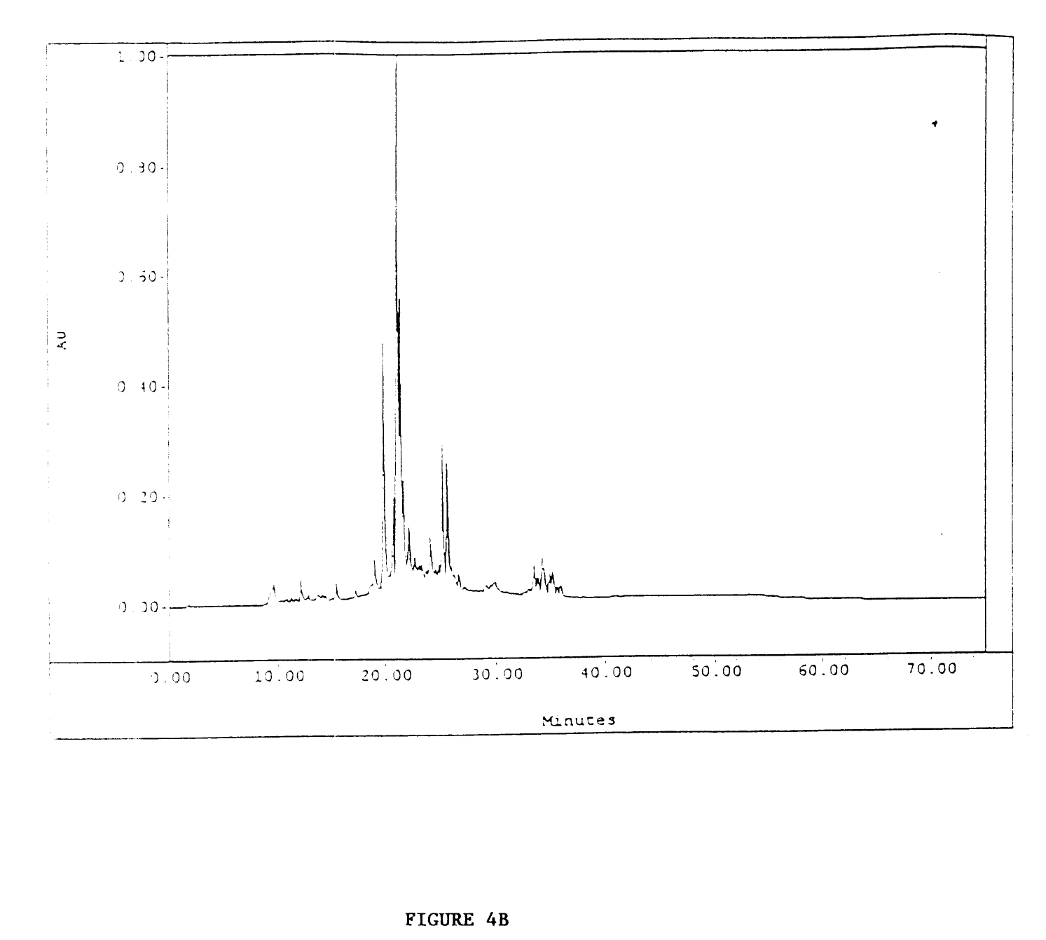

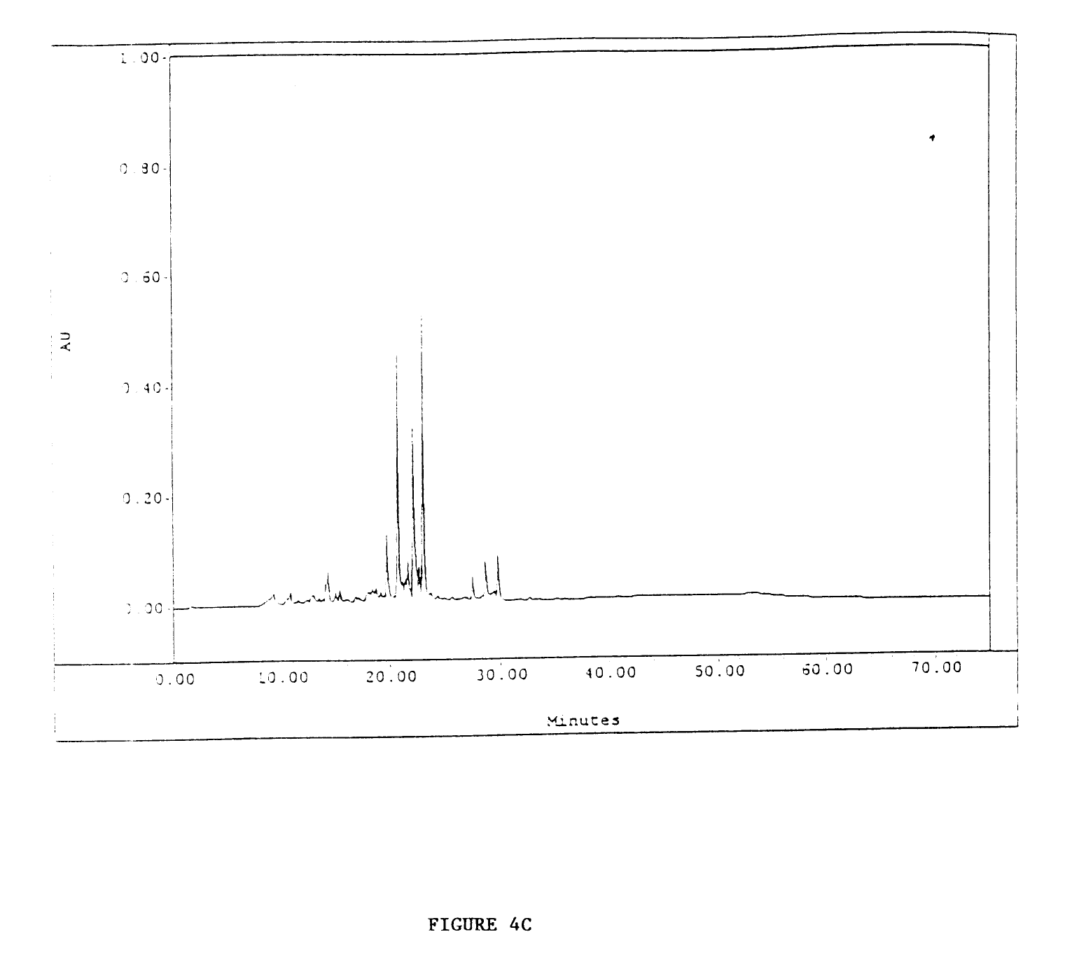

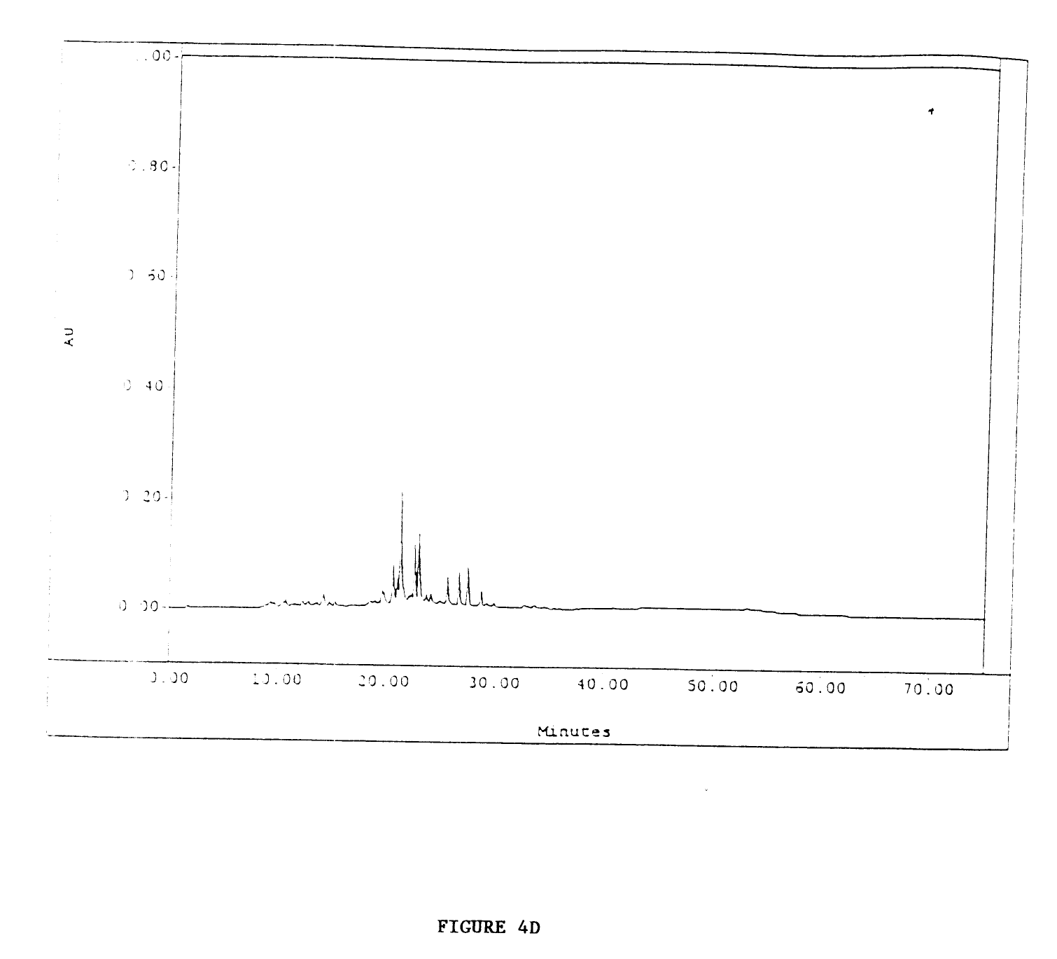

Ungerminated E. californica seeds were either pretreated by exposure to 5-azacytidine, or not pretreated. After uniform germination, callus induction and subculture, suspension initiation, and suspension subculture, the resulting suspension subcultures were subjected to T1 (control) and T3 conditions. After organic solvent extraction of the cell biomass, HPLC analysis demonstrated that the pretreatment by exposure to a DNA methylation inhibitor such as 5-azacytidine significantly altered the size, number, and location of peaks in the chromatograms (See FIG. 4). These data show that pretreating the seed with a DNA methylation inhibitor affects the production of both constitutive (no elicitor exposure) and inducible (elicitor exposure) metabolites.

### Example 4

HCV ATPase Inhibition

HCV ATPase inhibition was assayed in a microtiter-plate format by a modification of the procedure of Suzich et al. The reaction mixture contained 50 mM MOPS (pH 6.5), 1.95 mM phosphoenolpyruvate, 100μg/mL pyruvate kinase, 25 μg/mL lactate dehydrogenase, 100 μg/mL NADH, 2.5 mM MgCl₂, 1 mM ATP, and 5 μg/mL cloned HCV ATPase (clone NS3b, obtained from Dr. Darryl Peterson, Medical College of Virginia) in a total volume of 100 μL. The reactions were monitored continuously at 340 nm for 20 minutes, and initial velocity was determine by fitting a curve to the data. Selected extracts were found to have greater inhibitory activity than control extracts.

### Example 5

DNA/RNA Polymerase Inhibition

Assays for HIV reverse transcriptase and calf thymus DNA polymerase α were performed as described by August et al., and HSV-2 DNA polymerase as described by Elion et al., except that the reactions were run in microtiter plates and [α-³²P]TTP was used in place of [³H]TTP. The reactions were terminated by the addition of equal volumes of 10% trichloroacetic acid and allowed to stand on ice for 15 min. The precipitates were then transferred to glass-fiber filter mats using a Tomtec Harvester 96, and incorporated radioactivity was determined by liquid scintillation counting. Selected extracts were found to have greater inhibitory activity than the controls.

### Example 6

Cytotoxicity and Antifungal Activity

The ability of a particular extract to inhibit the growth of U937 cells in culture or the growth of Candida albicans was determined by growing the organisms in the presence of extract, and determining the viability of the culture relative to an untreated culture by the XTT method as described by Roehm et al. Similarly, the ability of an extract to inhibit the growth of Vero cells was determined by Sulforhodamine B method (Sigma Chemical Co.) according to the manufacturer's instructions. Selected extracts demonstrated significant anti-fungal activity and desirable cytotoxic characteristics.

### Example 7

HIV Protease Inhibition

Recombinant HIV-1 protease at a concentration of 1 μg/mL was incubated with 5 μM synthetic substrate (7-methoxycoumarin-4-yl) acetyl-GSQNYPIVGK(2,4-dinitrophenyl)-CONH₂), 0.1 M sodium acetate (pH 4.7), 1 M NaCl, 1 mM EDTA, 1 mM DTT, and 1 mg/mL bovine serum albumin in a total volume of 100 μL. The incubation was carried out at 37° C. for 20 min. and the reaction was terminated by adding 10 μL of 1 M sodium acetate (pH 4.0). The fluorescence was read at excitation wavelength 328 nm, emission wavelength 421 nm, in a Perkin Elmer LS-50B luminescence spectrometer equipped with a plate reader. Ac-Thr-lle-Nleψ(CH₂NH)Nle-Gln-Arg-NH₂ was used as a positive control. See also, Knight, C. G., et al., (1992) FEBS Letters, 296:163-266. Matayoshi, E. D., et al. (1990) Science, 247:954-958. Selected extracts were found to have greater inhibitory activity than control extracts.

### Example 8

CMV Protease Inhibition

Purified cytomegalovirus (CMV) protease expressed in E. coli was assayed using a synthetic fluorescent substrate (7-methoxycoumarin-4-yl)acetyl-RGVVNASSRLAK(2,4-dinitrophenyl)K-COOH). The reaction mixture (30 μL total) contained 1 μM CMV protease, 30 AM synthetic substrate, 0.1. M MOPS (pH 7.2), 0.1 mg/mL bovine serum albumin, and 10% glycerol and was incubated at 37° C. for 30 min. The reaction was terminated by the addition of 120 μL sodium acetate (pH 4.0). The fluorescence was read at excitation wavelength 328 nm, emission wavelength 416 nm, in a Perkin Elmer LS-50B luminescence spectrometer equipped with a plate reader. Zinc chloride was used as a positive control. Selected extracts were found to have greater inhibitory activity than control extracts.

### Example 9

Stability to Cryopreservation

Suspension cultures of the present invention are placed in pre-growth medium containing an osmoticum or osmoprotectant (e.g., mannitol, sorbitol, or glucose) to reduce the water content of the cells and vacuoles. This reduction, in turn, ameliorates the damage caused by internal ice crystals upon freezing. The concentration of the osmoticum generally is in the range between 0.5 M and 0.75 M. After 3-4 days of pregrowth at 25° C., batches (e.g., 1 gram) of cells are harvested from the cultures and placed in cryogenic vials. Added to each vial is a cryoprotectant mixture containing an independently selected osmoticum (e.g., a mixture containing DMSO, proline, and glycerol). The vials are incubated over ice-water for 1 h. Freezing is a two-stage process, such as 10 min at 0° C., slow freezing to −35° C. at the rate of −1° C./min, then 40 min at −35° C. followed by rapid freezing and storage in liquid nitrogen.

Thawing is also a progressive process, carried out rapidly (e.g., +9° C./min) in a warm water bath. The contents of the vial are placed carefully on filter paper, which is placed on agar medium. Cryoprotectants are typically removed by frequent transfer of cells to clean filters. Medium components such as activated charcoal are used to absorb toxins. After 3 days in the dark at 25° C., the filter is transferred to fresh medium, and observations are recorded. Successful storage results in renewed growth as callus cultures following 28 days. From these cultures, suspension cultures are reinitiated, subcultured, exposed to an elicitor system, and analyzed in terms of their chemical profile and pharmacological activities.

### OTHER EMBOIDMENTS

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method of affecting seccondary metabolite production in a plant culture, comprising
(a) exposing a liquid plant culture to a first DNA methylation inhibitor;
(b) subculturing said DNA methylation inhibitor-exposed liquid plant culture at least three times;
(c) exposing said subculture to an elicitor system; and
(d) maintaining said elicitor system-exposed liquid culture.

2. A method of claim 1, further comprising after step (b) and before step (c), the further step of exposing said subculture to a second DNA methylation inhibitor.

3. A method of claim 1, wherein said liquid plant culture is a plant cell suspension culture.

4. The method of claim 3, wherein each of said first and second DNA methylation inhibitiors is independently selected form 5-azacytidine, 5-aza-2′-deoxycytidine, 5-fluorocytidine, pseudoisocytidine, DL-ethionine, and 2-amino-5-ethoxycarbonylpyrimidine-4(3H) one.

5. A metthod of claim 4, wherein each of said first and second DNA methylation inhibitiors is 5-azacytidine.

6. A method of claim 3, wherein said elicitor system has at least one elicitor, each elicitor being independently selected from microorganism-derived elicitors, plant-derived elicitors, and chemically-defined elicitors.

7. A method of claim 6, wherein said each elicitor is independently selected from methyl jasmonate, salicylic acid, glutathione, 2,6-dichloroisonicotinic acid, cellulase, chitosan, chitin, nigeran, arachidonic acid, peroxide cascade intermediates, and elicitors derived from Candida albicans, Saccharomyces cerevisiae, Aspergillus niger, Phytophthora cryptogea, Pseudomonas syringae, and Erwinia caratovora pv. carotovora.

8. A method of claim 3, wherein said subculturing step is subculturing said liquid culture at least twice.

9. A method of claim 1, wherein said liquid culture is a differentiated liquid plant culture selected from embryo, root, shoot, hairy root, and teratoma.

10. A method of claim 9, wherein each of said first andsecond DNA methylation ihibitors is indpendently selected from 5-azacytidine, 5-aza-2′-deoxycytidine, 5-fluorocytidine, pseudoisocytidine, DL-ethionine, and 2-amino-5-ethoxycarbonylpyrimidine-4(3H) one.

11. A method of claim 10, wherein each of said first and second DNA methylation inhibitors is 5-azacytidine.

12. A method of claim 9, wherein said elecitor system has at least one elicitor, each elicitor being independently selected microorganism-derived elicitors, plant-derived elicitors, and chemically-defined elicitors.

13. A method of claim 12, wherein said each elicitor is independently selected from methyl jasmonate, salicylic acid, glutathione, 2-6-dichloroisonicotinic acid, cellulase, chitosan, chitin, nigetan, arachidonic acid, peroxide cascade intermediates, and elicitors derived from Candida albicans, Saccharomyces cerevisiae, Aspergillus niger, Phytophthora cryptogea, Pseudomonas syringae, and Erwinia caratovora pv. carotovora.

14. A method of affecting the secondary metabolite production profile in a plant culture, comprising
(a) exposing an ungerminated seed to a first DNA methylation inhibitor;
(b) deriving tissu from said DNA methylation inhibitor-exposed seed;
(c) initiating a callus culture from said derived tissue;
(d) subculturing said initiated callus culture;
(e) initiating suspension from said callus subculture; and
(f) maintaining said initiated suspension culture.

15. A method of claim 14, further comprising after said initiating step (e), the step of exposing a subculture of said suspension culture to a second DNA methylation inhibitor.

16. A method of claim 14, wherein each of said first and second DNA methylation inhibitors is independently selected from 5-azacytidine, 5-aza-2′-deoxycytidine, 5-fluorocytidine, pseudoisocytidine, DL-ethionine, and 2-amino-5-ethoxycarbonylpyrimidine,-4-(3H) one.

17. A method of claim 16, wherein each of said first and second DNA methylation inhibitors is 5-azacytidine.

18. A method of claim 14, wherein said exposing step (a) comprises soaking the ungerminated seed in a solution of 5-azacytidine having a concentration between 3×10⁻⁶ and 3×10⁻⁴ M.

19. A method of claim 14, wherein said subculturing step (d) comprises subculturing said callus subculture at least two times.

20. A method of claim 14, wherein said maintaining step (f) comprises subculturing said suspension culture at least five times.

21. A method of claim 20, wherein said maintaining step (f) comprises subculturing said suspension culture at least ten times.

22. A method of claim 14, further comprising after step (f) the step of exposing said suspension culture to an elicitor system.

23. A method of claim 22, wherein said elicitor system has at least one elicitor, each elicitor being independently selected from microorganism-derived elicitors, plant-derived elicitors, and chemically-defined elicitors.

24. A method of affecting secondary metabolite production in a plant culture, comprising:
(a) exposting an ungerminated seed to a first DNA metylation inhibitor;
(b) deriving tissue form said DNA methylation inhibitor-exposed seed;
(c) initiating a culture from said derived tisue;
(d) exposing a subculture derived from said initiated culture to an elicitor system; and
(e) maintaining said elicitor-exposed subculture.

25. A method of claim 24, further comprising after said initiating step (c) the step of exposing a subculture derived from said initiated culture to a second DNA methylation inhibitor.

26. A method of claim 24, wherein each of said first and second DNA methlation inhibitors is independently selected from 5-azacytidine, 5-aza-2′-deoxytidine, 5-flurorcytidine, pseudoisocytidine, DL-ethionine, and 2-amino-5-ethoxycarbonylpyrimidine-4-(3H) one.

27. A method of claim 26, wherein each of said first and second DNA methylation inhibitors is 5-azacytidine.

28. A method of claim 24, wherein said exposing step comprises soaking the ungerminated seed in a solution of 5-azacytidine having a concentration between 3×10⁻⁶ and 3×10⁻⁴ M.

29. A method of claim 24, wherein
said deriving step (b) comprises initiating a callus culture and subculturing said callus culture at least twice,
said initiating step (c) is initiating a suspension culture from said secondary callus subculture, and further comprising after step (c), the step of subculturing said suspension culture at least once, before said elicitor-exposing step (d).

30. A method of claim 24, wherein said culture of step (c) is a differentiated liquid culture selected from embryo, root, shoot, hairy root, and teratoma.

31. A method of claim 24, wherein said elicitor system has at least one elicitor, each elicitor being independently selected from microorganism-derived elicitors, plant-derived elicitors, and chemically-defined elicitors.

32. A method of claim 31, wherein said each elicitor is independently selected from methyl jasmonate, salicylic acid, glutathione, 2,6-dichloroisonicotinic acid, cellulase, chitosan, chitin, nigeran, peroxide cascade intermediates, and elicitors derived from Candida albicans, Saccharomyces cerevisiae, Aspergillus niger, Phytophthora cryptogea, Pseudomonas syringae, and Erwinia caratovora pv. carotovora.
